(19) United States
(12) Patent Application Publication
(10) Pub. No.: US 2026/0289773 A1
(43) Pub. Date: Sep. 24, 2026

(54) SYSTEMS AND METHODS FOR CORNEAL OPACITY DETECTION USING OPTICAL COHERENCE TOMOGRAPHY

(71) Applicant: Oregon Health & Science University, Portland, OR (US)

(72) Inventors: David Huang, Portland, OR (US); Yan Li, Portland, OR (US); Jad F. Assaf, Portland, OR (US); Jiachi Hong, Portland, OR (US)

(21) Appl. No.: 19/574,325

(22) Filed: Mar. 21, 2026

Related U.S. Application Data
(60) Provisional application No. 63/775,613, filed on Mar. 21, 2025.

(51) Int. Cl. G06T 7/00; A61B 3/10; A61B 3/117; G06T 7/11; G06T 7/514; G06T 7/521

(52) U.S. Cl. CPC G06T 7/0012; A61B 3/102; A61B 3/1176; G06T 7/11; G06T 7/514; G06T 7/521; G06T 2207/10101; G06T 2207/30041

(57) ABSTRACT

Methods and systems for detecting opacity in the cornea using optical coherence tomography are described. Corneal reflectance is analyzed as a function of anatomic layer within the cornea, depth within a given anatomic layer, and the angle of incidence of the OCT beam on the anterior corneal surface. Also disclosed is a thresholding technique based on the percentile distribution of reflectance values from a retrospective analysis of OCT scans from normal corneas. The techniques described herein are not dependent on the specific hardware configuration of the OCT system used, and they allow the automated detection, analysis, and quantitative characterization of corneal opacities and provide insight into tissue directionality as a function of depth within specific corneal layers.

### CROSS-REFERENCE TO RELATED APPLICATIONS

This application claims the benefit of U.S. provisional patent application No. 63/775,613, filed Mar. 21, 2025, titled “Systems and Methods for Corneal Opacity Detection Using Optical Coherence Tomography,” which is incorporated herein by reference.

### ACKNOWLEDGEMENT OF GOVERNMENT SUPPORT

This invention was made with government support under R01 EY029023 and R01 EY028755 awarded by The National Institutes of Health. The government has certain rights in the invention.

### TECHNICAL FIELD

This invention relates to methods and systems for detecting corneal opacities using optical coherence tomography (OCT).

### BACKGROUND INFORMATION

Corneal opacity is a clinically significant cause of visual loss. It can result from trauma, infection and dystrophies. Traditionally, diagnosing and evaluating corneal opacity relies on subjective assessment with the slit lamp biomicroscope, which require considerable skills to manually operate and much experience and training to derive accurate diagnosis from. Given the challenges associated with the variable reflectance of corneas and subjective nature of existing assessment methods, there is a substantial need for an objective and standardized diagnostic approach.

Optical coherence tomography (OCT) offers an alternate modality to evaluate the cornea by delivering high-resolution, cross-sectional images that enable detailed visualization of corneal pathology beyond what is possible with conventional slit-lamp techniques. OCT-based methods can identify the extent and severity of corneal scars, dystrophies, and other pathologies with greater consistency. However, reflectance in OCT images of the cornea can vary considerably with anatomic layer, depth within the corneal thickness, and incidence angle of the OCT scanning laser. Conventional OCT analyses often assume uniform or simplified reflectance properties of the imaged tissues, which is unsuitable for corneal applications. Thus there is a need for systems and methods to improve OCT-based imaging and analysis of the cornea that takes into account the significant variation in corneal reflectance associated with the various corneal anatomic layers, the depth within those layers, and the wide range of incidence angle of the OCT probe beam on the corneal surface.

### SUMMARY OF THE DISCLOSURE

Systems and methods for detecting and quantifying cornea opacities using OCT are disclosed. The disclosed systems and methods include a model-based approach that incorporates anatomic layer segmentation, depth dependence, and the incidence angle of the OCT beam. The use of such a reflectance model mitigates variability in clinical interpretation and provides a means to establish adaptable layer-specific threshold cut-off levels from population data to address different clinical scenarios or severity level. By establishing normative reflectance behavior with layer-, depth- and incidence angle-dependence, corneal regions exhibiting abnormally high reflectance may be identified as potential opacities. For example, threshold criteria such as the 97th and 99.9th percentile may be used to capture suspect and definite opacities, respectively. However, practice of the disclosed systems and methods is not limited to these, or any, specific thresholds and can be adapted to utilize different percentile levels or values to accommodate various clinical settings or specific medical conditions.

The disclosed systems and methods confer several advantages over the prior art, including reduced subjectivity in the interpretation of clinical findings, and compatibility with commonly available OCT systems. The disclosed systems and methods identify opacities on OCT images and can be used to plan treatments to remove the opacity by mechanical means or laser ablation. Furthermore, the disclosed systems and methods provide quantitative metrics to characterize extent of opacity regions in a patient, and enables the clinician to track progression or improvement of the corneal condition over time.

In some embodiments, the systems and methods may comprise receiving or acquiring an OCT dataset of an imaged cornea and segmenting the cornea into two or more anatomic layers (e.g., the epithelium, stroma, and endothelium). Each of these anatomic layers may further be divided into a series of depth increments spanning the anterior-to-posterior thickness of a given anatomic layer. Each pixel within a given anatomic layer is associated with depth within that layer and with an angle of incidence as measured between the direction of the A-scan to which that pixel belongs and the surface normal at the anterior corneal surface upon which the OCT scan beam is incident. A parameterized reflectance model having anatomic layer-, depth-, and angle of incidence-dependence and derived from a sample population of clinically normal corneas is used to identify pixel values exceeding a specified threshold reflectance value. In a typical embodiment, the reflectance model may be characterized by a set of parameters that are specific to each depth increment within a given anatomic layer. In a particular embodiments as described above, a first threshold level may be used to identify a set of suspect opacity pixels within the cornea, and a second, larger threshold may be used to identify a set definite opacity pixels. Together, these two pixel sets may be used to generate an opacity map for use in guiding clinical decision-making and treatment.

Additional aspects and advantages will be apparent from the following detailed description of preferred embodiments, which proceeds with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Applicant considers the color versions of the drawings as part of the original submission and reserves the right to present color images of the drawings in later proceedings.

To easily identify the discussion of any particular element or act, the most significant digit or digits in a reference number refer to the figure number in which that element is first introduced.

FIG. 1 shows an exemplary OCT radial scan pattern and corresponding cross-sectional image of a cornea.

FIG. 2A shows 2D OCT B-scan image of a cornea illustrating the in-plane incident angle calculations.

FIG. 2B shows an exemplary 3D model of a cornea illustrating out-of-plane incident angle calculations and showing an off-vertex cross-section B-scan.

FIG. 3 is a plot showing a comparison of incidence angle calculation on an OCT B-scan using a 2D approach with a single cross sectional image versus a 3D methodology using corneal elevation data.

FIG. 4 shows an exemplary plot of the absolute mean reflectance (log scale) vs. depth in corneal layers.

FIG. 5 shows an exemplary plot of absolute mean reflectance as a function of incidence angle for different corneal layers.

FIG. 6 shows an exemplary plot of absolute 97th percentile reflectance vs. depth in corneal layers, averaged over incidence angles of 1-23°.

FIG. 7 shows an exemplary plot of absolute 97th percentile reflectance as a function of incidence angle for different corneal layers.

FIG. 8 shows an exemplary plot of absolute 99.9th percentile reflectance vs. depth in corneal layers, averaged over incidence angles of 1-23°.

FIG. 9 shows an exemplary plot of absolute 99.9th percentile reflectance as a function of incidence angle for different corneal layers.

FIG. 10 shows a set of corneal OCT images, each with a corresponding mask overlay of identified regions of opacity.

FIG. 11A shows a table of results for the mean directional reflectance components of corneal layers. The percent reflectance of the directional reflectance component is the area under the curve of each exponential component divided by the sum area-under-the-curve of all exponential components, computed from incidence angles 1 to 23°.

FIG. 11B shows a table of results for the directional reflectance components of corneal layers at the 97th percentile.

FIG. 12A shows an exemplary plot of the absolute mean reflectance (log scale) vs. depth in corneal layers.

FIG. 12B shows an exemplary plot of absolute mean reflectance as a function of incidence angle for different corneal layers.

FIG. 12C shows an exemplary plot of absolute 97th percentile reflectance vs. depth in corneal layers, averaged over incidence angles of 1-23°.

FIG. 12D shows an exemplary plot of absolute 97th percentile reflectance as a function of incidence angle for different corneal layers.

FIG. 13A shows an image of 6-mm wide cornea OCT B-scan in log scale with enhanced contrast for improved visualization.

FIG. 13B shows the image of FIG. 13A converted to color scale to better illustrate the reflectance values.

FIG. 14 schematically shows an example system for detecting corneal opacities in accordance with the disclosure.

FIG. 15 schematically shows an example of a computing system in accordance with the disclosure.

FIG. 16 shows a cross sectional OCT image of a healthy cornea illustrating the influence of OCT beam incidence angle and tissue depth on corneal reflectance.

FIG. 17 shows a set of images depicting the fusion of 97^{th} and 99.9^{th} percentile masks using morphological operations on an OCT B-scan from a patient with corneal dystrophy. The final detected opacity mask is shown in red.

FIG. 18 shows an example of a set of representative corneal OCT B-scans demonstrating automated opacity detection across three pathologies: monoclonal gammopathy (top row), Salzmann nodular degeneration (middle row), and granular corneal dystrophy (bottom row). Left column: original OCT image. Middle column: overlay of the automated opacity mask (red) and the consensus manual annotation (blue), defined as agreement by ≥3 of 5 annotators; areas of overlap appear in yellow. Right column: inter-annotator agreement heatmap, with each pixel color-coded by the number of annotators who marked it as opacity (dark blue=1, light blue=2, green=3, yellow=4, orange=5).

FIG. 19 shows an example of algorithm-based opacity detection in a case of macular corneal dystrophy. The opacity mask (shown in red) reveals diffuse stromal and Descemet's opacification. Such cases highlight the need for customized opacity thresholds to better guide PTK planning by targeting the most significant opacities.

FIG. 20 shows an exemplary method in flowchart form that may be used to practice certain aspects of the methods described herein.

FIG. 21 shows a flowchart of a method for generating a reflectance model from population data in accordance with the methods described herein.

### DETAILED DESCRIPTION OF EMBODIMENTS

Corneal opacity is an important clinical finding, characterized by the loss of corneal transparency, which can result from diverse etiologies such as trauma, infection, edema, and various dystrophies. Clear vision depends on the cornea's ability to transmit light with minimal scatter, which in turn is linked to the uniform arrangement of collagen fibril s in the stroma. Disruption of this arrangement increases light scatter, producing opacity. Large-scale registry data indicate that 6.5% of patients in the United States were diagnosed with corneal opacity, with corneal dystrophies as the most frequent underlying cause, underscoring the prevalence of this condition. Despite its impact on vision, the assessment of corneal opacity often remains subjective, relying on slit-lamp biomicroscopy, which depends heavily on clinical expertise. This subjectivity underscores the need for an objective, standardized method.

Disclosed herein are systems and methods for detecting corneal opacities using OCT. The disclosed systems and methods assess corneal reflectance across multiple corneal anatomic layers and across multiple angles of incidence of the OCT scanning beam to determine the presence and severity of opacities with high precision and reliability. The disclosed systems and methods may also be used to measure the layer- and depth-wise variations in corneal reflectance to establish ranges of normal (non-pathologic) reflectance values for use as a reference to define threshold values or threshold functions for detecting various aberrations in the cornea, including opacities. The characterization of layer- and depth-wise reflectance in the cornea may further be used to infer underlying microstructure, such as directionality of collagen fibrils or fibril density, of the substituent imaged tissue.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

In order to facilitate review of the various embodiments of the disclosure, the following explanation of specific terms is provided:

A-scan: A reflectivity profile that contains information about spatial dimensions and location of structures within an item of interest. An A-scan is directed along the optical axis of the OCT device and penetrates the sample being imaged. The A-scan encodes reflectivity information (for example, signal intensity) as a function of depth. When the term “reflectance” is used herein, it generally refers to the fraction of light reflected from the cornea along an A-scan as measured by an OCT device.

B-scan: A cross-sectional tomograph that may be achieved by laterally combining a series of axial depth scans (i.e., A-scans). A B-scan encodes planar cross-sectional information from the sample and is typically presented as an image.

OCT Dataset: As used herein, an OCT dataset is an ordered-array representation of stored data values that encodes relative spatial location in row-column-depth (x-y-z) format. In the context of OCT, as used herein, a dataset may be conceptualized as a three dimensional array of voxels, each voxel having an associated value (for example, an intensity value or a reflectance value). An A-scan corresponds to a set of collinear voxels in the depth (axial) direction of the dataset; a B-scan is made up of set of adjacent A-scans combined in the lateral (row) direction. Such a B-scan may also be referred to as an image, and its constituent voxels referred to as pixels. A collection of adjacent B-scans can be combined in the vertical (column) direction to form a 3D volumetric set of voxel data (or 3D image). The most basic form of an OCT dataset as used herein is a single A-scan. More typically, however, a dataset is comprised of multiple A-scans organized into one or more B-scans.

Optical coherence tomography (OCT) is an optical signal acquisition and processing method that is capable of capturing micrometer-resolution, two- and three-dimensional images from within optical scattering media, e.g., biological tissue. Optical coherence tomography is based on interferometric techniques and typically employs near-infrared light. The use of relatively long wavelength light allows it to penetrate into the scattering medium. As remarked above, among its many applications, OCT-based ocular imaging has found widespread clinical use and can be performed quickly and easily with minimal expertise. OCT is a non-invasive imaging modality which, in the context of the present disclosure, is able to provide accurate and precise anatomical reproduction of the cornea and it different anatomic layers, and thus is well suited for use in detecting and diagnosing pathologic conditions within the cornea.

The systems and methods disclosed herein employ a reflectance model that accounts for normal physiologic and anatomic variations in the cornea. In a preferred embodiment, a three-part reflectance model may be formulated to incorporate corneal anatomic layer, depth within a given corneal anatomic layer (which may be expressed as a percentage), and the incidence angle of the OCT laser scan. Aspects of a three-part reflectance model include:
Layer Boundaries: Rather than assume uniformity of tissue reflectance properties in the cornea, the reflectance model accounts for the presence of multiple anatomic layers which can have different reflective properties. Accordingly, the model may be formulated to include multiple corneal layers, including, for example, layers corresponding to epithelium, Bowman's layer, stroma (which may be further divided, for example, into anterior, middle, and posterior zones or sublayers), and endothelium.
Depth Within Layer: The disclosed model may be formulated to account for reflectance changes as a function of position (depth along an A-scan) from anterior to posterior within a given layer.
Incidence Angle: Because reflectance varies according to the angle of incidence formed between the OCT beam and the cornea surface normal, the model may be formulated to account for this light-tissue interaction.

When formulated in this three-part manner, a mathematical model of reflectance enables an automated determination of whether a region of high reflectance represents normal variation or pathological opacity. For example, by fitting acquired measurement data from a population of eyes to a mathematical model to capture variations in corneal reflectance as functions of depth and angle of incidence, it is possible to establish robust thresholds for identifying corneal opacities. Such mathematical models can include linear, piecewise linear, spline, and non-linear models to accommodate the complex nature of the data. In a particular embodiment described in examples below, a mathematical model comprising exponential functions is used to fit the reflectance measurement data across multiple different corneal layers. As will be shown below, such a reflectance model formulation may be used to derive or fit model coefficients to data to define, for example, the mean reflectance across different corneal layers. Similarly, model coefficients may be fit to, for example, binned data values to establish threshold functions at different offsets from the mean—such as 97th, and 99.9th percentile thresholds—to aid in determination of outlier reflectance values that may be indicative of localized opacity or other conditions. It should be noted that while an exponential model formulation is used in the examples below, it is to be understood that the specific choice of model can vary based on the specific needs and characteristics of the data to be fit.

The systems and methods described herein are compatible with a range of OCT technologies, encompassing swept-source (SS), and spectral-domain (SD) OCT, as well as advanced techniques such as hyperparallel (HP) OCT, full-field OCT, and line field OCT. This compatibility extends to various scanning protocols, accommodating radial scans, raster scans for a grid-like mapping of the cornea, and spiral scanning patterns. For example, while the examples below utilized eight radial cuts (see Example 1, Example 2, and Example 4), it is to be understood that the scanning and analysis methods can be adapted to include different numbers of radial cuts, such as 6, 12, or 24, depending on the specific requirements of the diagnostic task. Similarly, beam spacing for raster and spiral scans can be adapted to meet the requirements of the specific diagnostic task.

OCT devices are commonly calibrated using diffuse reflectance targets. Such calibration approaches ensure consistency and comparability of reflectance values across devices. For example, standardized diffuse reflectance targets (e.g., 12%, 25%, or 50% reflectance panels) are well known in the art and may be used with the methods disclosed herein to ensure the reproducibility and reliability of the described techniques across different OCT machines. By way of example, a standardized calibration technique, utilizing standard diffuse reflectance targets with varying reflectance rates (12%, 25%, and 50%) may be used to map the squared magnitude of the OCT signal to absolute reflectance values. Such an approach allows one to infer the magnitude corresponding to 100% reflectance by application of linear regression analysis.

It is noted that while standardized diffuse reflectance targets typically include a 99% reflectance panel, it can be advantageous in the practice of the disclosed systems and methods to omit this panel due to the saturation it produces at the detector of the OCT device. In various applications and embodiments, it can also be advantageous to convert OCT reflectance magnitudes into absolute reflectance values by dividing them by the magnitude representative of 100% reflectance. This conversion can be important for normalizing the data, enabling the generalization of the disclosed methods to function effectively across various OCT machines, regardless of their inherent magnitude variations.

Additional preprocessing of OCT data may include signal roll-off compensation to address depth-dependent signal attenuation in OCT datasets. For example, signal roll-off compensation may be applied to every image in an acquired OCT dataset to correct for the reduction in signal strength with increasing depth.

Data preprocessing in the form of speckle-attenuation may also be employed using various techniques known in the art, for example, by application spatial filters to reduce noise and improve image quality. Examples of such filters include, but are not limited to, a low pass mean filter, Gaussian filter, median filter, bilateral filter, non-local means filter, box filter, Kalman filter, and Weiner filter.

In order to preprocess OCT data for use in a layer- and depth-wise mathematical reflectance model, layer segmentation procedures may be implemented as part of the present disclosure. Accordingly, various boundary detection methods known in the art may be used to delineate by depth different layers of the cornea captured in the OCT images. For example, the cornea may be segmented in epithelium, stroma, and endothelium layers through the depth of the corneal thickness. In some embodiments, it may be advantageous to further subdivide a given layer into two or more sublayers. The stroma, for example, may be divided into anterior stroma, middle stroma, and posterior stroma layers.

As discussed above, the proposed mathematical reflectance model includes association of reflectance measurements at each pixel or voxel of an A-scan with a corresponding incident angle. The incident angle can be defined as the angle between the cornea surface normal vector and the OCT beam direction vector. Anterior corneal surface elevation data of the OCT scan, may be used to define the surface normal, using either a 2D or 3-D representation of the anterior corneal surface. The direction of each A-scan in combination with the surface normal direction may be used to calculate incidence angle for each A-scan. In some embodiments, the angle of refraction at the at the air-cornea interface may also be used to calculate the incidence angle, as further described in Example 1 and Example 2 below.

In the methods disclosed herein, a variety of image processing techniques are employed to identify and categorize pixels that may contribute to regions of opacity within the cornea. Pixels that surpass pre-determined intensity thresholds, as calculated from depth-dependent and angle-specific reflectance models that have been fit to population data for a representative sample of OCT data, are flagged for further analysis. The specific levels of these thresholds are adjustable depending on the specific requirements and sensitivity desired for the opacity detection and the details of the reflectance model and population data used to fit the reflectance model. For example, thresholds may be defined at various percentiles within the range of population data. In the Examples below, the 97^{th} and 99.9^{th} percentiles are used to classify pixels as either “suspect” or “definite” opacities. However, it will be appreciated by those having skill in the art that other numerical ranges, distributions, or indices may be used.

In various embodiments, 2D and 3D morphological operations may be employed to enhance the robustness of opacity detection with pixel or pixel groupings. For example, morphological operations such as dilation and merging may be used to refine and consolidate identified opacity regions into a unified opacity mask. As part of this processing methodology, in an initial step, smaller clusters of opacity pixels that do not meet a minimum connectivity criterion may be excluded to focus on larger or more significant and clinically relevant opacity areas. These operations and transformations may be flexibly applied depending on specific clinical requirements and characteristics of the patient-specific data, and may be applied in multiple dimensions, accommodating various scanning protocols, including raster and spiral scans. The methodology may also be practiced in a manner that allows for the simultaneous use of multiple threshold values or threshold functions (e.g., multiple threshold percentiles) to combine different types of opacities, each opacity type meeting distinct criteria for inclusion. Furthermore, once detected, regions of corneal opacity may be quantified in terms of area, volume, intensity, or other metrics. Such quantification may be employed in a single imaging session or may be employed over multiple imaging sessions to monitor progression of the opacity defect over time or to track response treatment or other intervention.

In a particular embodiment, a systematic “pixel grouping” approach may be used to assess the reflectance properties of the cornea by grouping adjacent pixels based on shared optical characteristics. This pixel grouping approach enables a detailed analysis of the cornea's reflective behavior across its various anatomical layers, and is especially beneficial when used in concert with automatic layer segmentation algorithms provided by the OCT system or any layer segmentation approaches known in the art. As a specific example of anatomic layers that may be segmented into separate groups for analysis, a first corneal layer—the epithelium—extending from the anterior corneal boundary to the Bowman's membrane, may be distinguished from a second corneal layer—the stroma—spanning from Bowman's membrane to the corneal posterior boundary.

An important aspect of the proposed pixel grouping method is that it enables the efficient capture and analysis of the variations in corneal reflectance as a function of incidence angle and depth change, which is critical for identifying and assessing corneal opacities. Central signal flares in A-scans, known to distort the reflectance, can be excluded during pixel grouping to improve data quality and downstream analysis. Furthermore, groups of pixels from the same corneal layer imaged from different patients can be aggregated based on their depth and angle of incidence. This approach allows for the synthesis of data from equivalent anatomical positions across a patient cohort population, thereby enabling a more comprehensive and statistically robust analysis for describing the range of angle- and depth-dependent variation in corneal reflectance. For example, analysis may include grouping reflectance values into bins based on depth and angle of incidence. Within each bin, defined by its layer depth and angle of incidence, a percentile analysis may be performed—such as calculation of the mean, 97^{th}, and 99.9^{th} percentiles—to establish criteria for detecting different levels of corneal reflectance or for use as a threshold criterion to detect corneal opacity. By aggregating pixels into bins and employing detailed reflectance analysis, improved statistical significance may be realized and the noise typically associated with individual pixel analysis is reduced.

Another important aspect of the proposed pixel grouping method is that it can be implemented in manner independent of the pixel size used by different OCT systems. This pixel size-independence ensures that the method can be utilized across different OCT devices and technologies, regardless of their inherent pixel resolutions.

### Example 1

The following Example 1 provides a demonstration of how the disclosed systems and methods might be applied to detect opacities in OCT-imaged corneas. Here we present a model-based approach that incorporates layer segmentation, depth dependence, and the incidence angle of the OCT beam. By establishing normative reflectance behavior under these conditions, corneal regions exhibiting abnormally high reflectance are flagged as potential opacities. Threshold criteria such as the 97th and 99.9th percentile can be used to capture suspect and definite intensity opacities, respectively.

Methods: OCT Data and Scan Pattern: We analyze OCT scan data, focusing on corneal OCT B-scan images to assess corneal reflectance and detect opacities. OCT data was obtained from the Visionix (formerly Optovue) Avanti platform (North Lombard, IL, USA). While the methods and analysis described in herein may be applied using any OCT and scan pattern, a radial scan pattern used to conduct this particular Example, utilizing a radial scan pattern that produces 8 radial 6-mm OCT B-scans.

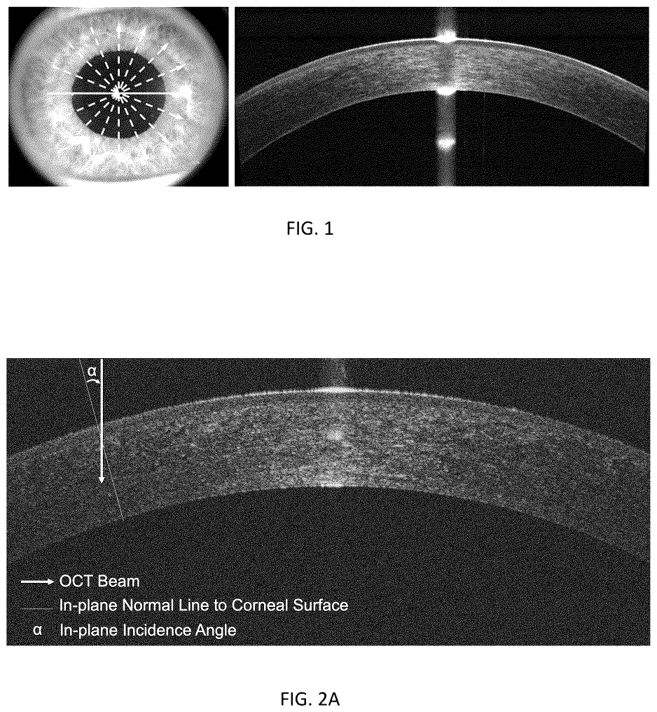

FIG. 1 shows an exemplary OCT radial scan pattern as used in this Example and a corresponding cross-sectional image. As shown, the OCT radial scan pattern (left) utilized to obtain the data, highlights the alignment of the eight radial lines along meridians with the corresponding cross-sectional OCT B-scan (right). This representation showcases how each B-scan corresponds to specific radial meridian lines. The scan pattern is centered on the pupil, often slightly offset from the corneal vertex, aligning with common clinical imaging practices.

Patient Selection: Control patients classified as normal and pre-LASIK were included, confirmed through a comprehensive slit lamp examination with no abnormal corneal findings. Patients with previously identified corneal opacities, verified during a slit lamp examination, were included to validate the opacity detection algorithm. Exclusion criteria were strictly applied to patients who had undergone corrective laser ablation procedures, as these treatments could alter corneal characteristics and potentially confound the study's analysis of intrinsic corneal properties.

OCT Calibration: The calibration of OCT systems is a critical step in ensuring the reproducibility and reliability of the algorithm across different OCT machines. To achieve this, we employed a standardized calibration technique, utilizing standard diffuse reflectance targets with varying reflectance rates (12%, 25%, and 50%) to map the squared magnitude of the OCT signal to absolute reflectance values. This approach allowed us to infer the magnitude corresponding to 100% reflectance through linear regression analysis. It's noteworthy that we opted not to use the panel with 99% reflectance directly in our measurements due to the saturation of the detector.

Subsequently, the OCT magnitudes were converted into absolute reflectance values by dividing them by the magnitude representative of 100% reflectance. This conversion is essential for normalizing the data, enabling the generalization of our algorithm to function effectively across various OCT machines, regardless of their inherent magnitude variations.

Data Preparation: In the data preparation phase, eight radial cut images were extracted from each OCT scan, maintaining their raw, non-processed, and non-de-warped format. The choice to utilize non-de-warped images was deliberate, aiming to ensure that each column (A-scan) accurately represented the true path of light rays as they traverse the cornea. The image pixels corresponded to the reflectance of the light signal in the linear domain at any given point. We applied a signal roll-off compensation for every image, a technique to correct for the reduction in signal strength with increasing depth in OCT imaging, as well as a low-pass mean filter for speckle attenuation.

Incidence Angle Calculation: The incident angle is defined as the angle between the surface normal and the OCT beam in air. To accurately calculate the incident angles for our analysis, we utilized anterior corneal surface elevation data to compute the surface normals. We then proceeded to calculate the incident angle for each A-scan for a 3D calculation of incident angles.

A 2D calculation of the angle would assume that the highest point of the B-scan is the corneal vertex, with an out-of-plane incidence angle of zero. The out-of-plane incidence angle is the angle between the OCT beam and the surface normal in the plane perpendicular to the B-scan. This assumption is only valid if the B-scan passes through the vertex, which is rarely the case. As a result, 2D calculations are often inaccurate for determining incident angles and a 3D approach is required.

In this implementation, while the external incident angle has been primarily employed to model corneal reflectance, a similar relationship can also be established with the angle of refraction. The angle of refraction is defined as the angle formed between the corneal surface normal and the refracted OCT beam at the air-cornea interface.

A 3D OCT scan comprises a series of cross-sectional B-scans made of parallel A-scans aligned within a plane. In a raster scan, the B-scans are arranged in a parallel stack. For the scanning of a dome shaped structure such as the cornea, the B-scans can also be arranged in a radial spoke fashion (radial scan) centered on an ocular landmark such as the pupil, corneal limbus, or corneal vertex reflection. In standard corneal OCT scans, the incident OCT beam remains parallel to the optical axis of the OCT system during scanning in a telecentric fashion. Once the OCT beam encounters refractive tissue boundaries, its direction changes. But here we are referring to the beam direction just prior to the point of incidence on the first tissue boundary. Since the gaze of the eye is also fixated on a visual target coaxial to the OCT optical axis, the optical axis is perpendicular to the cornea at the vertex. Because the B-scans do not intersect exactly with the vertex in general, the 3D incidence angle θ between the OCT beam and the corneal surface normal (the geometric line drawn perpendicular to the anterior corneal surface) generally has two orthogonal components: in-plane incidence angle α within the plane of the B-scan and out-of-plane incidence angle β in the plane in the plane perpendicular to the B-scan. Angle α is the angle between the incident OCT beam (A-scan axis) and the projection of the surface normal within the plane of the B-scan. Angle β is the angle between the incident OCT beam and the projection of the surface normal in the plane perpendicular to the B-scan but parallel to the A-scan. Angle α is measured by tracing the orientation of the anterior corneal surface the B-scan image. Angle β is measured from a 3D model of the anterior corneal surface contour compiled from multiple B-scans. The 3D incidence angle θ is calculated by combining α and β in a root sum square fashion.

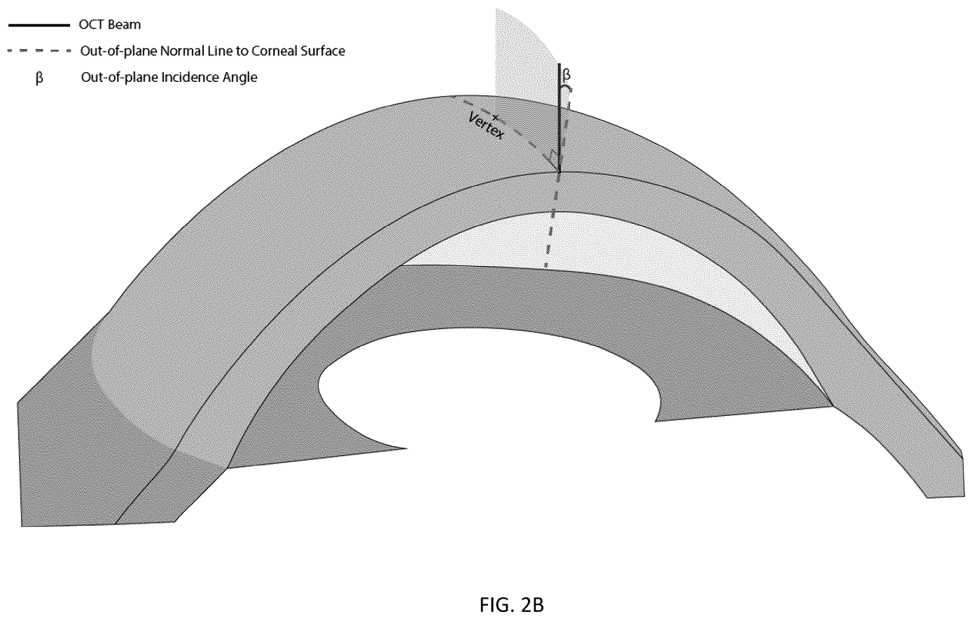

FIG. 2A shows a cross-sectional OCT image to illustrate calculation of incidence angle from a single 2D B-scan. In this case, incidence angle α is the angle between the incident OCT beam (i.e., the A-scan axis) and the surface normal within the plane of the B-scan.

FIG. 2B shows a 3D model illustrating 3D incident angle calculations on the cornea, showing an off-vertex cross-sectional B-scan. In standard corneal scanning, the eye is fixating on a visual target that is parallel with the optical axis of the OCT system and the telecentrically scanned OCT beam. Thus, the OCT beam is parallel to the corneal vertex line. The B-scan plane, traced by the OCT beam, is the cut-away shown, and is the focus of our analysis. The OCT beam is at an incidence angle θ with respect to the line perpendicular to the anterior corneal surface, which is also known as the surface normal. The incidence angle θ has two components: in-plane incidence angle α within the plane of the B-scan (as depicted in FIG. 2B) and out-of-plane incidence angle β in the plane in the plane perpendicular to the B-scan. While angle α is easily measured using information in the 2D B-scan, a 3D model of the anterior corneal surface constructed from multiple B-scans is necessary to compute angle β. Once both α and β a determined, the 3D incidence angle is calculated according to θ=\sqrt{a²+β²}. This illustration demonstrates the necessity of considering in and out of plane angles for accurate 3D incidence angle computation.

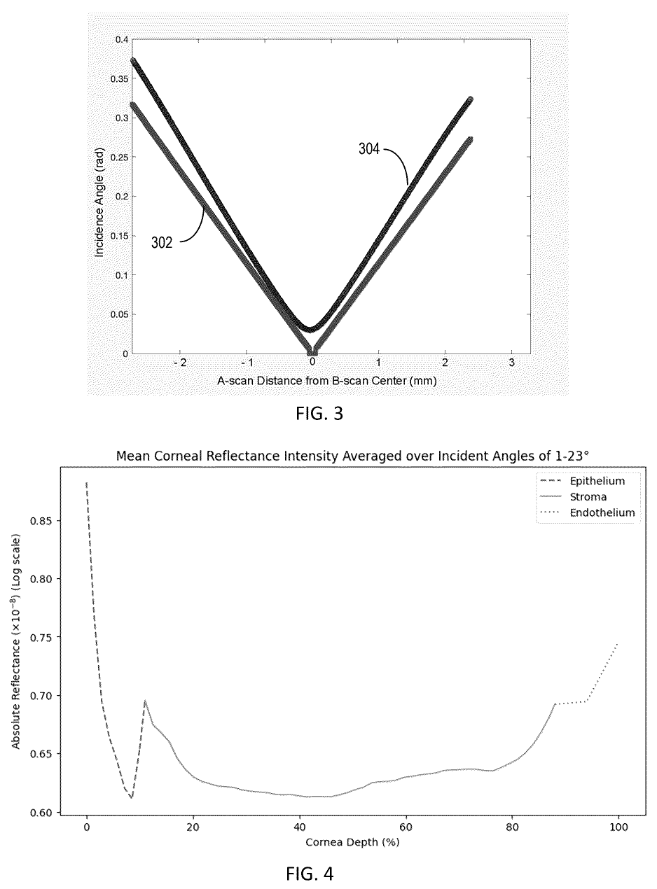

FIG. 3 illustrates the advantages of our method by contrasting the incidence angle calculations performed using conventional 2D methods—based solely on the OCT B-scan and assuming the vertex is the highest point—with those derived from our 3D method using elevation data. Specifically, FIG. 3 shows an exemplary plot generated from a selected OCT B-scan and comparing incidence angle calculations obtained using a 2D approach (302) and a 3D methodology incorporating corneal elevation data (304). The x-axis represents A-scan position relative to the center of the selected B-scan, and the y-axis represents the corresponding calculated incidence angle in radians. In this example, the 2D incidence angle values were derived from the anterior segmentation of the selected B-scan using a 2D surface normal, whereas the 3D incidence angle values were derived using corneal elevation data to estimate a 3D surface normal at corresponding locations. The plotted profiles are scan-specific and may vary depending on the selected B-scan and its position relative to the corneal apex.

Pixel Grouping Analysis: In our methodology, we develop a systematic approach to assess the reflectance properties of the cornea by grouping adjacent pixels based on shared optical characteristics. This technique, which we will refer to as “pixel grouping,” enables a detailed analysis of the cornea's reflective behavior across its various anatomical layers, which are segmented automatically using algorithms provided by the OCT system. Specifically, the epithelium extends from the anterior corneal boundary to the Bowman's membrane, while the stroma spans from Bowman's membrane to the corneal posterior boundary.

The purpose of this pixel grouping method is to efficiently capture and analyze the variations in corneal reflectance with respect to incidence angle and depth change, critical for identifying and assessing corneal opacities. Central signal flares in A-scans, known to distort the reflectance, were excluded from the analysis. Furthermore, groups of pixels from the same corneal layer across different patients were aggregated based on their depth and angle of incidence. This approach allows us to synthesize data from equivalent anatomical positions across a diverse cohort, enhancing the comprehensiveness and reliability of our analysis. Within each bin, defined by its layer depth and angle of incidence, we perform percentile analysis—such as the mean, 97^{th}, and 99.9^{th}—to establish criteria for detecting different levels of corneal opacity.

Importantly, our methodology is designed to be independent of the pixel size used by different OCT systems. This generalizability ensures that our approach can be seamlessly applied across various OCT technologies, regardless of their inherent pixel resolutions. By aggregating pixels into bins and employing detailed reflectance analysis, we improve the statistical significance of our findings and reduce the noise typically associated with individual pixel analysis.

For visualization and clarity in presentation, the cornea was subdivided into various anatomic layers for detailed analysis:
Epithelium: Distinguished into middle and posterior sections.
Bowman's Layer: Identified as the region surrounding the transition between the epithelium and stroma.
Stroma: Segmented into anterior, middle, and posterior regions.
Endothelium and Descemet's Membrane: Recognized as the innermost layer adjacent to the cornea's posterior boundary.

Curve Fitting: In our approach, we use mathematical models to capture variations in corneal reflectance as functions of depth and angle of incidence, thereby establishing robust thresholds for identifying corneal opacities. Mathematical models can include linear, piecewise linear, spline, and non-linear models to accommodate the complex nature of the data. In the Example 1 presented here, we use exponential functions to fit the reflectance models derived from the mean, 97th, and 99.9th percentile thresholds across different corneal layers. The choice of model can vary based on the specific needs and characteristics of the data.

The general form of the exponential function applied for curve fitting is given by:

$${I}_{k} ( θ ) = {A}_{1 k} {e}^{( - {B}_{1} θ )} + {A}_{2 k} {e}^{( - {B}_{2} θ )} + {A}_{3 k} {e}^{( - {B}_{3} θ )} + C$$

Where:
I represents the intensity at incremental depth layer k,
θ is the angle of incidence of light on the cornea, measured in radians.
A₁ₖ, A₂ₖ, and A₃ₖ are the amplitude coefficients for the exponential terms tailored to the k-th layer, indicating the maximum reflectance magnitude.
B₁ₖ, B₂ₖ, and B₃ₖ are the decay constants for each exponential term in the k-th layer, describing how reflectance decreases as the angle of incidence increases.
C is the constant capturing baseline reflectance.

This flexible framework allows for adjustments and optimizations to accurately reflect the specific optical properties of each layer. It is noted that in the above formulation, the curve fitting process yields a unique set of fitting coefficients and constants for each depth increment within a given anatomic layer. For example, a division of 10 depth increments through the thickness of the epithelium will yield 10 sets of fitting parameters (constants and coefficients) to describe the depth-wise reflectance behavior of the tissue.

To enhance the continuity and stability of our model across different corneal depths, regularization techniques are applied to penalize large variations in the parameters between successive depth layers, smoothing the transition of parameters and improving the robustness of the mode.

Thresholding Technique: In our methodology, image processing techniques are employed to identify and categorize pixels that potentially represent opacity regions within the corneal structure. Pixels surpassing pre-determined intensity thresholds, derived from depth-dependent and angle-specific reflectance models, are flagged for further analysis. These thresholds are adaptable and can be set at various percentiles, such as the 97th and 99.9th percentiles, depending on the specific requirements and sensitivity desired for the opacity detection.

To enhance the robustness of our opacity detection, we utilize a comprehensive image processing framework that includes both 2D and 3D morphological operations. This framework integrates techniques such as dilation and merging to refine and consolidate identified opacity regions into a unified opacity mask. Initially, smaller clusters of opacity pixels that do not meet a minimum connectivity criterion are excluded to focus on more significant and clinically relevant opacity areas. These transformations are specifically designed to be flexible and can operate in multiple dimensions, accommodating various scanning protocols, including raster and spiral scans. The methodology allows for the simultaneous use of multiple threshold percentiles to combine different types of opacities, each meeting distinct criteria.

In this specific implementation, regions displaying signal intensities above the 97th percentile are logically combined with those surpassing the more stringent 99.9th percentile threshold, using operations such as:

resultant_region=Dilate(upper_threshold_region)Λlower_thresholdregion

Additionally, larger identifiable segments within the lower threshold mask are isolated and integrated into the final comprehensive reflectance mask, representing the areas of corneal opacity, using operations such as:

unifiedMask=resultant_region+LargeSegments(lower_threshold_region,20)

The composite mask can then be analyzed to assess the area, intensity, and precise location of opacities within the cornea.

Results: Mean Reflectance Analysis: The mean reflectance models for each corneal layer demonstrated high accuracy with an R-Square average of 0.987, indicating a strong fit to the observed data.

The mean epithelium model was fitted with a single exponential component, highlighting its less directional behavior:

$$I ( θ ) = {A}_{epi} {e}^{( - Bepi θ )}$$

The mean stroma and endothelium models were each fitted using two exponential components to capture their complex reflectance dynamics:

$$I ( θ ) = {A}_{1} {e}^{( - {B}_{1} θ )} + {A}_{2} {e}^{( - {B}_{2} θ )}$$

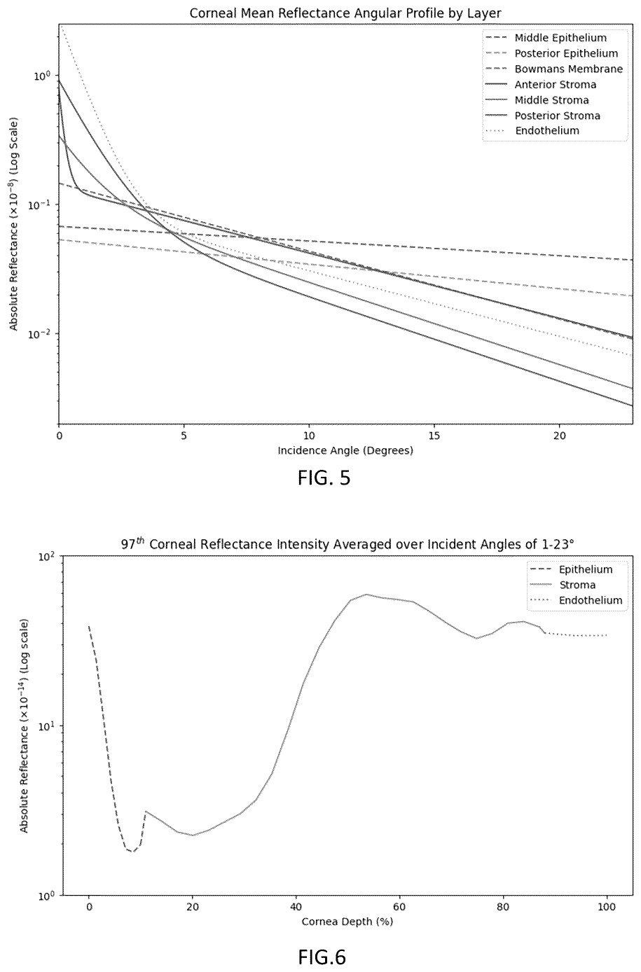

FIG. 4 shows a plot of the absolute mean corneal reflectance (log scale) versus depth in corneal layers, averaged over incident angles of 1 to 23°. We note the high reflectance of the anterior and posterior stroma compared to the middle stroma. FIG. 5 shows a plot of the absolute mean reflectance (log scale) as a function of the incidence angle for different corneal layers. While the epithelium is less directional than the remaining layers in the cornea, there exists intra-layer directionality variations within the stroma itself. The anterior stroma is the most directional layer.

97^{th} and 99.9^{th} Percentile Reflectance: For the epithelium, the reflectance data were best modeled using two exponential functions in conjunction with a constant C, effectively capturing the layer's reflectance variations at both the 97th and 99.9th percentiles.

$${I}_{epi} ( θ ) = {A}_{1 epi} {e}^{( - B 1 epi θ )} + {A}_{2 epi} {e}^{( - B 2 epi θ )} + C$$

Conversely, both the stroma and endothelium layers demonstrated a more complex reflectance pattern, necessitating the use of three exponential functions alongside a constant C to accurately model the data across the same percentiles.

$${I}_{k} ( θ ) = {A}_{1 k} {e}^{( - B 1 k θ )} + {A}_{2 k} {e}^{( - B 2 k θ )} + {A}_{3 k} {e}^{( - B 3 k θ )} + C$$

This differentiation in the number of exponentials required reflects the distinct directionality and optical properties inherent to each corneal layer.

For each of the 97^{th} and 99.9^{th} percentile models, we plotted the corneal reflectance averaged over incident angles and the directionalities for different corneal layers (FIGS. 6-9). The middle and posterior stroma have higher average reflectance than the anterior stroma. The anterior stroma is still highly directional compared to the rest of the corneal layers.

FIG. 6 shows a plot of the absolute 97^{th} percentile reflectance vs. depth in corneal layers, averaged over incidence angles of 1-23°

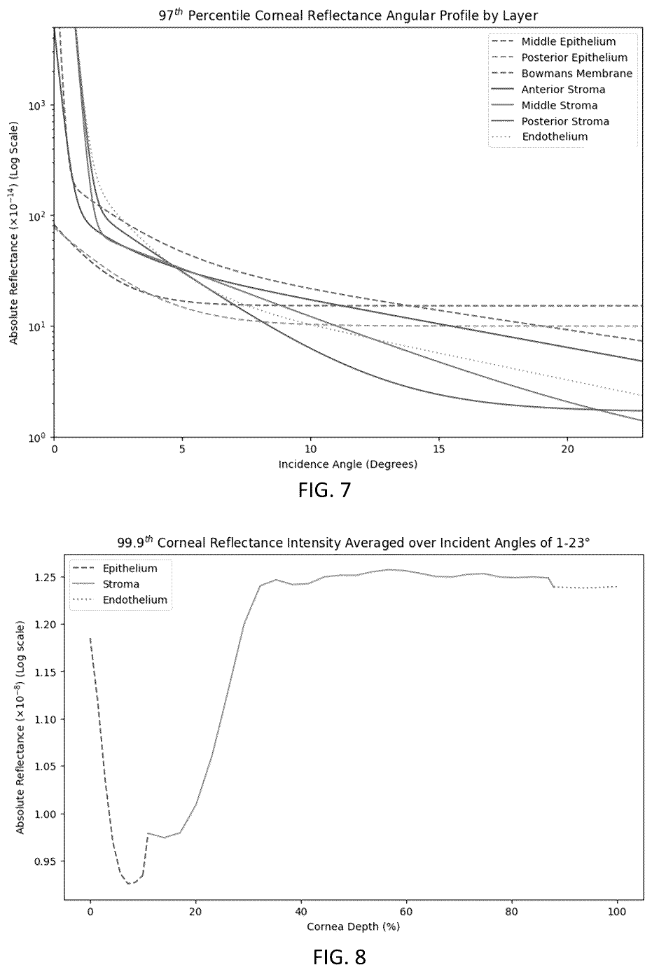

FIG. 7 shows a plot of the absolute 97^{th} percentile reflectance as a function of incidence angle for different corneal layers.

FIG. 8 shows a plot of the absolute 99.9^{th} percentile reflectance vs. depth in corneal layers, averaged over incidence angles of 1-23°.

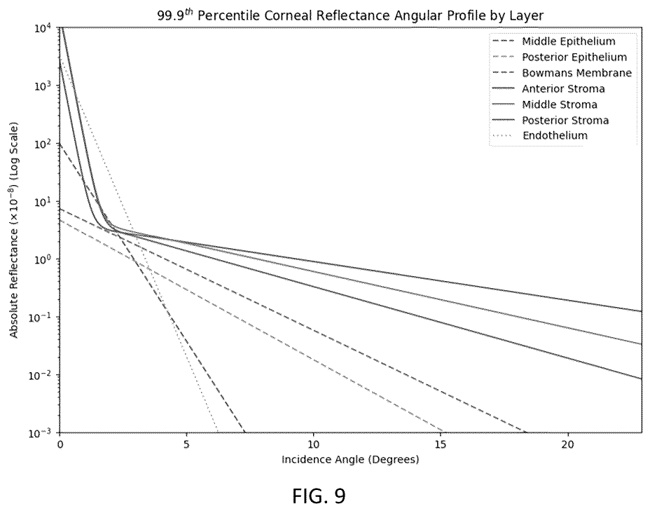

FIG. 9. shows a plot of the absolute 99.9^{th} percentile reflectance as a function of incidence angle for different corneal layers.

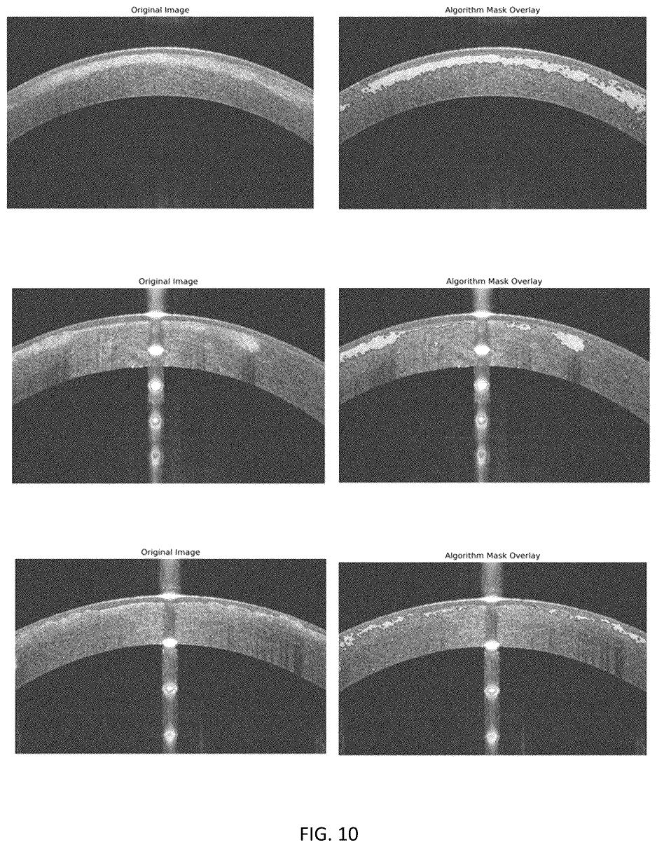

FIG. 10 shows examples of automated opacity detection from patients with (top row) monoclonal gammopathies (multiple myeloma) and (middle and bottom rows) granular dystrophy. These examples highlight the algorithm's capability to identify and segment opacities.

Conclusions: By incorporating layer boundaries, depth variation, and incidence angle in a baseline reflectance model, the disclosed systems and methods more reliably detect corneal opacity in OCT images than methods assuming uniform reflectance. The incorporation of percentile-based thresholds enables flexible identification of mild versus severe opacities, while standard methods of calibration, segmentation, and morphological processing refine the final maps. The result is a more objective and standardized approach for evaluating corneal opacity, potentially aiding clinical decision-making and treatment planning.

### Example 2

The following Example 2 provides a demonstration of how the disclosed systems and methods might be applied to establish layer-specific threshold functions using a normative data analysis approach. This Example 2 also demonstrates how the disclosed systems and methods may be used to infer material directionality in different layers of the cornea.

Introduction: The cornea, a transparent, avascular tissue forming the anterior part of the eye, is fundamental in refracting and transmitting light onto the retina, thus playing a critical role in visual acuity. Composed primarily of organized collagen fibers and interspersed cellular elements, its structural integrity is essential for maintaining its optical clarity and function.

Optical coherence tomography (OCT) has emerged as a powerful, non-invasive imaging modality in ophthalmology, providing high-resolution, cross-sectional images of ocular structures, including detailed views of corneal layers. Beyond morphological assessment, OCT allows for the analysis of tissue optical properties through reflectance measurements. These reflectance profiles can offer valuable insights into tissue microstructure, scattering properties, and potential pathological alterations.

Previous seminal studies by Knighton et al. extensively characterized the directional reflectance properties of the retina, revealing significant relationships between retinal microstructure and reflectance patterns. These works highlighted how variations in tissue composition and organization influence light scattering and reflectance. Despite the cornea's importance, similar comprehensive analyses of its directional reflectance properties are lacking.

The cornea is susceptible to various pathologies such as keratoconus, corneal dystrophies, and scarring, which can disrupt its microarchitecture and alter its optical properties. Understanding the directional reflectance of the cornea could enhance the diagnostic capabilities of OCT by providing additional quantitative metrics related to corneal health and integrity. Such metrics could aid in the early detection of corneal pathologies, monitoring disease progression, and evaluating treatment outcomes, potentially informing novel diagnostic and therapeutic strategies.

Therefore, this study aims to characterize the directional reflectance of the cornea using OCT imaging. By analyzing reflectance across segmented corneal layers—the epithelium, stroma, and endothelium—and modeling reflectance as a function of layer depth and angle of incidence, we seek to develop a comprehensive model describing the cornea's reflectance characteristics. By bridging gaps in current understanding and exploring the corneal structure-function relationship, this study sets the stage for future investigations into the complex interplay between corneal structure and optical behavior.

Methods: This retrospective study was conducted at the Casey Eye Institute of Oregon Health & Science University (OHSU) and received approval from the OHSU Institutional Review Board (IRB). All procedures adhered to the tenets of the Declaration of Helsinki and complied with the Health Insurance Portability and Accountability Act of 1996. Patient data were anonymized to protect confidentiality.

Patient Selection: We analyzed OCT scans from a cohort of normal subjects to evaluate corneal reflectance properties. Informed consent was obtained from all subjects after a detailed explanation of the study's nature, procedures, and potential consequences. Inclusion criteria encompassed patients with 6-mm-wide OCT scans obtained using the Visionix (formerly Optovue) Avanti platform (North Lombard, IL, USA). Subjects were classified as normal or pre-LASIK candidates based on comprehensive ophthalmic examinations, including slit-lamp biomicroscopy, ensuring the absence of corneal abnormalities. Exclusion criteria included any history of corneal pathology, prior refractive surgery, or any ocular condition that could affect corneal reflectance.

OCT Imaging and Calibration: All OCT scans were performed using the Avanti OCT system, operating at a wavelength of 840 nm with an axial resolution of approximately 5 m. To ensure accurate reflectivity measurements, the OCT device underwent a rigorous calibration process. We utilized near-Lambertian diffuse reflectance targets (Spectralon®, North Sutton, NH) with known reflectance rates of 12%, 25%, and 50% to map the squared magnitude of the OCT signal to absolute reflectance values. A linear regression analysis was performed to establish the relationship between the OCT signal and the known reflectance standards, allowing for the estimation of the magnitude corresponding to 100% reflectance. The 99% reflectance panel was omitted from direct measurements to prevent detector saturation. Additionally, the reflectors' angle dependence was minimal, with less than +4% variation in reflectance observed over a 250 range of incidence angles.

Post-calibration, OCT signal intensities were converted to absolute reflectance values by normalizing against the calculated 100% reflectance magnitude. This standardization facilitates consistent data analysis across different OCT devices and imaging sessions.

Data acquisition and Preparation: For each subject, eight non-dewarped radial B-scan images were extracted from the OCT data. The non-dewarped images preserve the true optical representation of the cornea, ensuring that each A-scan corresponds accurately to the physical path of the OCT beam through the cornea. Pixel intensities in these images represent the reflectance of the tissue in the linear domain.

To correct for the inherent depth-dependent sensitivity loss and to mitigate speckle noise, we applied a depth-dependent signal roll-off compensation-derived from calibration with a diffuse reflectance target-followed by a low-pass mean filter. This processing pipeline rectifies the intensity data to yield accurate reflectance estimates that are generalizable to other devices.

Incidence Angle Calculation: Accurate determination of the angle of incidence for each A-scan was essential for modeling directional reflectance. We leveraged the OCT system's ability to image the curved cornea in vivo, which inherently provides a range of incidence angles due to the curvature of the cornea relative to the OCT beam. By assuming that the cornea is homogeneous at the same depths across the central and paracentral areas, we could utilize data from different regions to analyze reflectance at varying incidence angles without physically manipulating the light source or detector.

We employed a three-dimensional (3D) approach utilizing corneal surface elevation data to compute surface normals at each point on the anterior corneal surface. The incidence angle (θ) was calculated as the angle between the OCT beam vector in air and the normal vector to the corneal surface at each A-scan location.

This 3D method accounts for both in-plane and out-of-plane curvatures of the cornea, providing a more precise calculation of incidence angles compared to a two-dimensional (2D) method, which assumes that the B-scan intersects the corneal apex and neglect out-of-plane variations. FIG. 2 from Example 1 illustrates the differences between 2D and 3D incidence angle calculations on a sample OCT B-scan.

Corneal Layer Segmentation: The cornea was segmented into the epithelium, Bowman's layer, stroma, and endothelium/Descemet's membrane for analysis. The epithelium's posterior boundary was defined using the Avanti system's automated algorithm. Bowman's layer was defined as the 10 μm region just posterior to the epithelium, measured perpendicular to the local corneal tangent. The stroma, spanning from Bowman's layer to the endothelium, was further divided into anterior, middle, and posterior thirds. The endothelium and Descemet's membrane comprised the innermost 15 μm of the cornea. The anterior 25% of the epithelium was excluded from analysis due to the high reflectance from the air-tear interface. The variability of the tear film can affect the underlying anterior epithelial reflectance, and the limited axial resolution of the OCT system—defined by its point spread function—causes the strong reflectance from the air-tear interface to overlap with and obscure the reflectance from the underlying anterior epithelium.

Binning Procedure: To analyze reflectance properties systematically, each pixel in the OCT images was first associated with two parameters: corneal depth and the incidence angle. We then grouped the pixels into sets that fell within narrow, predefined ranges: 2% increments in corneal depth and 0.001 radians in incidence angle. This grouping effectively clusters the pixels and associated reflectance values into localized sets with nearly identical depth and angular characteristics, ensuring that the aggregated reflectance values accurately represent the optical behavior for that specific region of the cornea.

Pixel sets with similar parameters (depth and incidence angles) across all subjects in the dataset were then grouped together into bins. This binning strategy allowed us to aggregate sufficient data for mean reflectance analysis, which provides insight into the average reflectance changes across the cornea, capturing the overall optical behavior of each layer. Additionally, the 97^{th} percentile reflectance within each bin was assessed, reflecting higher-intensity scattering events that may indicate corneal opacities or subtle structural variations. The choice of a 2% depth increment and a 0.001 radian bin width was determined empirically to balance depth and angular resolution with the requirement for sufficient data in each bin.

Reflectance and Normality Testing: Before further analysis, we assessed the normality of reflectance values within each bin to determine the appropriate statistical methods for percentile extraction. Normality testing helps decide whether standard deviation can be used for extracting percentiles or if non-parametric approaches are necessary.

We performed normality testing for the reflectance of all pixels in the individual corneal layers—epithelium, stroma, and endothelium—before further analyzing bins. The D'Agostino-Pearson omnibus K-squared test was used, with a significance level (a) of 0.05. Layers or bins with p-values greater than 0.05 were considered to have normally distributed reflectance values, while those with p-values ≤0.05 were deemed non-normal.

The proportion of bins that passed or failed the normality test was recorded. We also visualized their distribution across the cornea to determine if normality patterns were associated with specific corneal regions or layers.

Curve Fitting and Reflectance Modeling: We modeled the mean and 97^{th} percentile reflectance profiles as functions of the incidence angle for all bins across the entire cornea. Inspired by Knighton et al. (1999)—who used an exponential function to describe directional reflectance in the retinal nerve fiber layer—we adopted a similar approach but allowed up to three exponential components to accommodate the more complex reflectance behavior in corneal layer.

To determine the appropriate number of components, we first fitted a single-exponential model. If R²>0.90, it was considered sufficient unless adding another component reduced ≥50% of the remaining gap toward a perfect fit (R²=1.0). This strategy ensured that additional complexity was only introduced when it provided meaningful improvement, thereby preventing overfitting. Moreover, this curve fitting approach enables extrapolation to angles of incidence not directly represented in our dataset, including large angles or near-zero angles subject to flare artifacts.

Afterwards, we grouped the results according to the predefined corneal layer segmentation (middle epithelium, posterior epithelium, Bowman's layer, anterior stroma, middle stroma, posterior stroma, and endothelium/Descemet's membrane) for analysis and presentation.

The general form of the exponential function used is:

$${I}_{k} ( θ ) = {A}_{1 k} {e}^{( - {B}_{1} θ )} + {A}_{2 k} {e}^{( - {B}_{2} θ )} + {A}_{3 k} {e}^{( - {B}_{3} θ )} + C$$

Where:
I represents the intensity at layer k, for incidence angle θ
θ is the angle of incidence of light on the cornea, measured in radians.
A₁ₖ, A₂ₖ, and A₃ₖ are amplitude coefficients representing the maximum reflectance magnitudes for the first, second, and third exponential components in layer k.
B₁ₖ, B₂ₖ, and B₃ₖ are decay constants describing how reflectance decreases with increasing incidence angle. The decay angle is the inverse of the decay constant B expressed as 1/B
C is the constant accounting for baseline reflectance.

Curve fitting was performed using non-linear least squares optimization in MATLAB R2023b (MathWorks Inc., Massachusetts, USA). Constraints were applied to ensure physically meaningful parameters:
A1k>A2k>A3k>0. The amplitude coefficients were constrained to ensure each subsequent component contributes less reflectance.
B₁ₖ, B₂ₖ, B₃ₖ≤0. Decay constants were constrained to be negative or zero, reflecting decreasing reflectance with increasing angle.
C≥0. The constant C was constrained to be non-negative, capturing the baseline reflectance.

Initial parameter estimates were based on preceding layers to promote continuity between layers. Regularization techniques were applied to penalize large parameter variations between adjacent depth layers, ensuring smooth transitions in the model.

The goodness of fit was evaluated using the coefficient of determination (R2), with values closer to 1 indicating a better fit.

Mean Variance Analysis: To quantify patient-level variability in corneal reflectance relative to our model, we performed a variance component analysis on the binned mean reflectance values. For each corneal layer, the residuals were computed as the percentage deviation between the measured mean reflectance of each binned pixel set and the model-predicted mean reflectance (linear domain). These normalized residuals were then partitioned into three variance components—between-patient, between-eye (within a patient), and within-eye—using standard one-way ANOVA.

In addition, we investigated potential demographic influences on reflectance variability. Pearson correlation analysis was performed to assess the association between patient age and the mean of residual variance for each layer. Furthermore, to evaluate gender differences, we compared the resulting distributions between male and female subjects using a two-sample t-test.

Results: Subject Demographics: A total of 160 OCT scans from 95 eyes of 49 patients were analyzed. The mean age of the subjects was 38.3±11.3 years (range 18-76 years), with 50% being male.

Data Distribution Normality: Reflectance values for the epithelium, stroma, and endothelium exhibited non-normal leptokurtic distributions with a right-tailed skew (p<0.0001). Specifically, 96.0% of bins in the epithelium (n=10,200), 98.2% in the stroma (n=51,000), and 74.3% in the endothelium (n=4,080) were non-normally distributed according to the D'Agostino-Pearson omnibus K-squared test. Approximately 3.6% of bins were found to be normally distributed, primarily at higher incidence angles where sample sizes are smaller and data are more susceptible to artifacts.

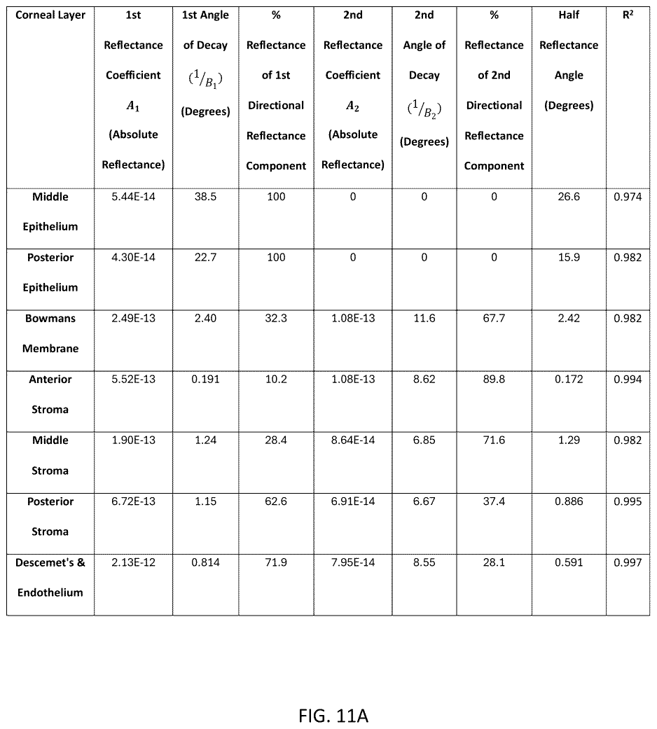

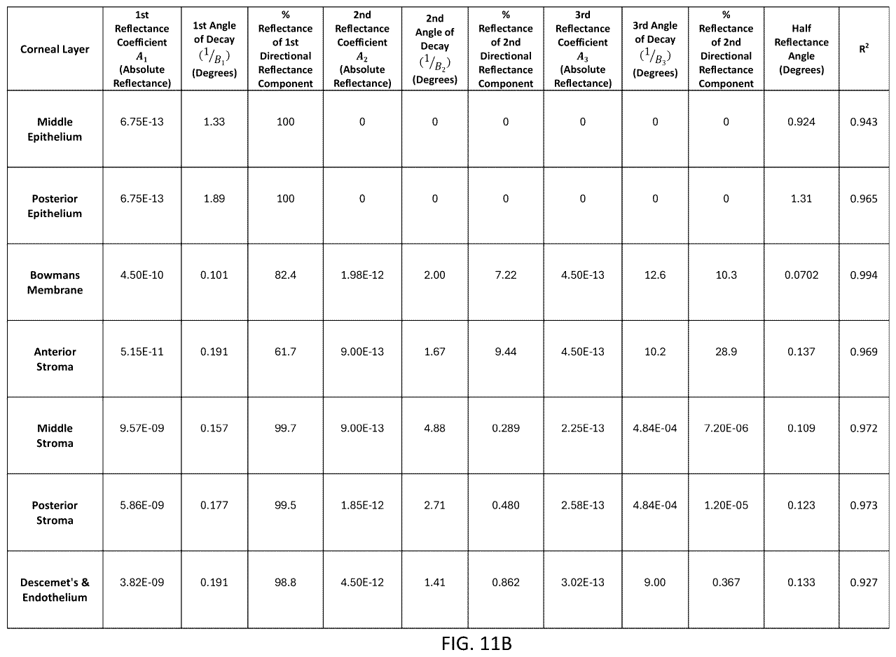

Reflectance Modeling and Curve Fitting: The mean and 97^{th} percentile reflectance models for each corneal layer were successfully fitted using exponential functions, demonstrating high goodness of fit with average R² values of 0.987 for mean reflectance and 0.963 for the 97^{th} percentile reflectance. The detailed parameters of the reflectance models for each corneal layer are presented in FIG. 11A and FIG. 11B.

Mean Reflectance Profiles: The mean reflectance of the epithelium was modeled by a single exponential function:

$$I ( θ ) = {A}_{epi} {e}^{( - Bepi θ )}$$

This indicates a less directional reflectance pattern compared to deeper corneal layers. The half-reflectance angles (the angle at which reflectance drops to half its maximum value) for the middle and posterior epithelium were approximately 26.58° and 15.85° (FIG. 11A), respectively, suggesting moderate angular dependence.

The stromal layers exhibited more complex mean reflectance patterns, requiring two exponential components for modeling:

$$I ( θ ) = {A}_{1} {e}^{( - {B}_{1} θ )} + {A}_{2} {e}^{( - {B}_{2} θ )}$$

Anterior Stroma: Demonstrated high reflectance and pronounced directionality, with the first (larger) component accounting for 10.2% of reflectance with a half-reflectance angle of 0.17°, indicating a sharp decrease in reflectance with increasing angle of incidence.

Middle Stroma: Showed lower overall reflectance, with the first component contributing 28.4% of reflectance and a half-reflectance angle of 1.29°.

Posterior Stroma: Exhibited high reflectance similar to the anterior stroma but with slightly less directionality (smaller angle of decay 1/B₁).

The Bowman's membrane, endothelium and Descemet's membrane also required a two-component exponential model, reflecting their unique structural composition.

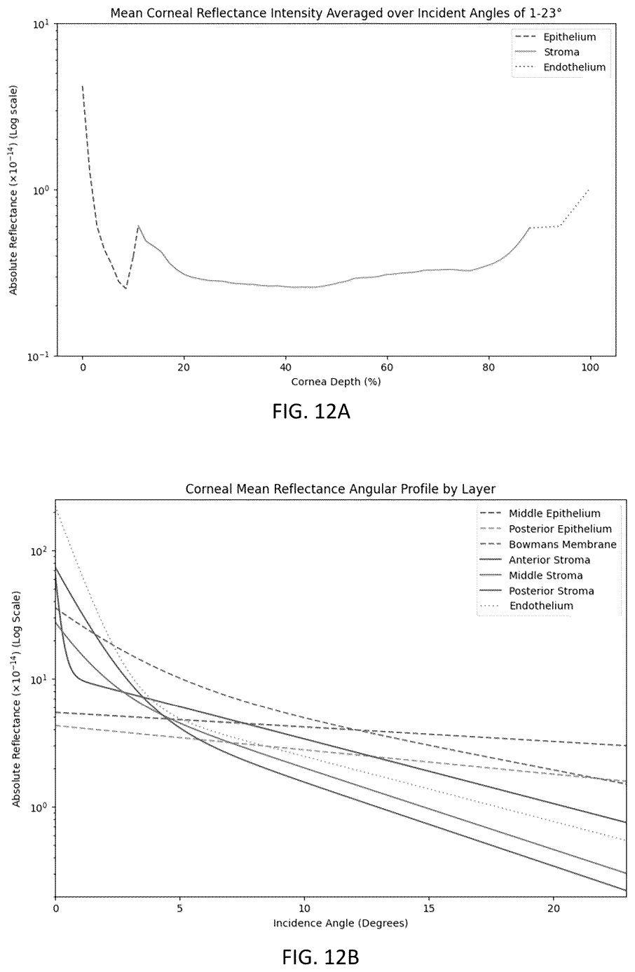

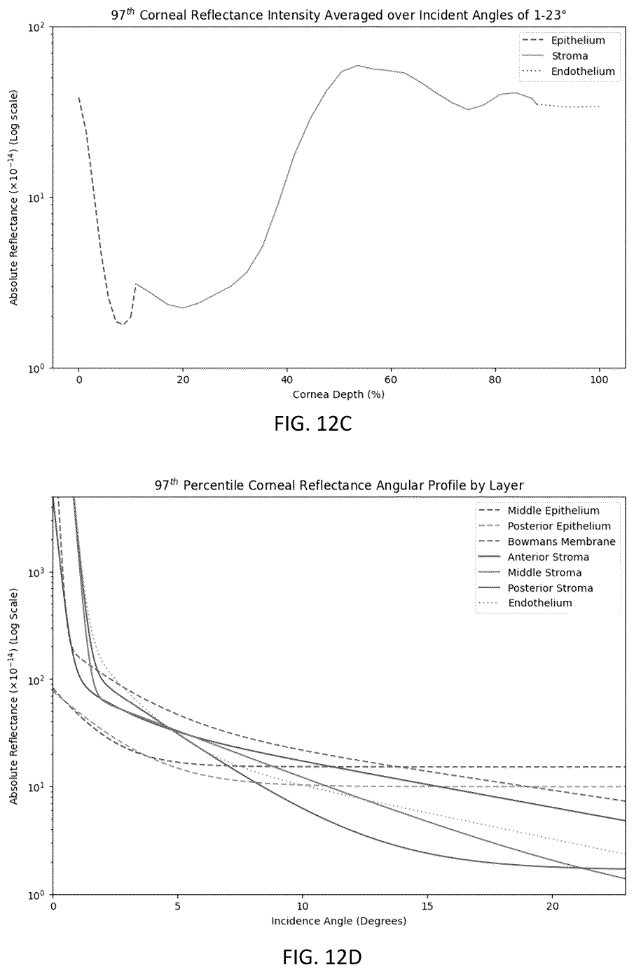

FIG. 12A and FIG. 12B illustrate the mean absolute reflectance as a function of corneal depth and incidence angle, respectively. The anterior and posterior stroma displayed higher reflectance compared to the middle stroma across most incidence angles.

97^{th} Percentile Reflectance Profiles: The 97^{th} percentile reflectance models and their detailed parameters are presented in tabular form in FIG. 11B.

The 97^{th} percentile reflectance profile of the epithelium was modeled as a single exponential function with a baseline constant C:

$$I ( θ ) = {A}_{epi} {e}^{( - Bepi θ )} + C$$

The Bowman's membrane, stroma, endothelium, and Descemet's membrane were modeled using 3 exponentials:

$$I ( θ ) = {A}_{1} {e}^{( - {B}_{1} θ )} + {A}_{2} {e}^{( - {B}_{2} θ )} + {A}_{3} {e}^{( - {B}_{3} θ )} + C$$

At the 97^{th} percentile, reflectance models (FIG. 12C and FIG. 12D) showed similar patterns to mean reflectance, but the middle and posterior stroma possess higher reflectance compared to the anterior stroma.

Mean Variance Analysis: Variance component analysis of the bin-level residuals revealed that, in the epithelium, 31.4% of the total normalized variance (overall variance=0.42) was attributable to differences between patients, 6.3% to differences between eyes, and 62.3% to within-eye variability. In the stroma, the variance was partitioned as 26.2% between-patient, 4.4% between-eye, and 69.5% within-eye (overall variance=0.56). For the endothelium, the components were 38.8% between-patient, 10.5% between-eye, and 50.7% within-eye (overall variance=0.49).

Pearson correlation analysis between patient age and normalized residual variance did not reveal any significant associations. For the epithelium, the correlation coefficient was r=−0.002 (p=0.992); for the stroma, r=−0.065 (p=0.667); and for the endothelium, r=−0.058 (p=0.700). Additionally, gender does not significantly affect corneal reflectance variability in the epithelium (p=0.523), stroma (p=0.176), and endothelium (p=0.177).

Discussion: This study provides a comprehensive analysis of the directional reflectance properties of the cornea using OCT imaging. By modeling reflectance as a function of incidence angle and corneal depth, we developed detailed exponential models for both mean and 97^{th} percentile reflectance profiles across different corneal layers. Our findings reveal distinct reflectance patterns and directionalities within these layers.

Reflectance values in the epithelium, stroma, and endothelium exhibited non-normal leptokurtic distributions with a right-tailed skew, necessitating the use of non-parametric techniques for percentile calculations. Approximately 3.6% of bins were normally distributed, likely due to statistical anomalies from the large number of bins tested. Most of these bins were at higher incidence angles where sample sizes are lower and data are subject to artifacts. At high incidence angles, the signal-to-noise ratio decreases, causing noise to overcome the signal and potentially leading to a more normal distribution.

Our results showed that the angular dependence of the mean reflectance of the middle and posterior epithelium is modeled by a single exponential function, indicating a less directional reflectance pattern. The half-reflectance angles were relatively large (26.580 and 15.85°), suggesting that reflectance decreases gradually with increasing incidence angle. This observation aligns with Rayleigh and Mie scattering theories, where small, randomly arranged scattering elements cause diffuse scattering with minimal dependence on the angle of incidence. The epithelial cells and subcellular structures are comparable in size to the OCT wavelength (840 nm), resulting in uniform reflectance across varying angles

In contrast, the stromal layers required two exponential components to model their mean reflectance, indicating the presence of multiple structural components, each with distinct angular dependence. The anterior and posterior stroma exhibited higher reflectance and more pronounced directionality compared to the middle stroma (FIG. 11A, FIG. 12A, FIG. 12B). The anterior stroma, with a half-reflectance angle of 0.17°, displayed the sharpest decrease in reflectance with increasing angle, indicating strong directionality. In comparison, the posterior and middle stroma had larger half-reflectance angles of 0.89° and 1.29°, respectively, reflecting a more gradual decline in reflectance and less pronounced directionality (FIG. 11A). However, the anterior stroma exhibited a less pronounced decrease in reflectance at lower incidence angles, indicating a flatter, less directional component compared to the middle and posterior stroma (FIG. 12B).

Two key structural factors that contribute to corneal transparency are the uniformity of collagen fibril diameters and the tight regulation of spacing between adjacent fibrils. Our findings suggest differences in collagen fiber organization, lamellar spacing, and keratocyte density across the stromal depth. Additionally, variations in tissue composition, such as the presence of a nerve plexus, may further contribute to these reflectance differences. According to Ruberti et al. (2011), the organization of the corneal lamellae displays a narrowed weaving pattern in the anterior stroma, whereas in the posterior stroma, the organization transitions to a plane pattern with lamellae lying regularly in the plane of the cornea. The intensity of reflectance is affected by refractive index variations within the tissue. Denser lamellae in the anterior stroma create more abrupt changes in refractive index, which can enhance reflectance intensity. The spacing between lamellae also influences reflectance; tightly packed lamellae can cause constructive interference of backscattered light, increasing reflectance, whereas regular spacing can promote destructive interference, contributing to corneal transparency. Differences in lamellar density, regularity, and spacing between stromal layers may explain why the middle stroma mean reflectance is less pronounced compared to the anterior and posterior stroma.

An interesting observation in our study is the distinct patterns of magnitude and directionality between the mean and 97^{th} percentile reflectance profiles. While the anterior and posterior stroma exhibited the highest mean reflectance, the 97^{th} percentile reflectance was much higher in the middle and posterior stroma (FIG. 12C, FIG. 12D). Despite this, the less directional component of the anterior stroma at higher incidence angles, observed in the mean reflectance, remains present relative to the middle and posterior stroma in the 97^{th} percentile profiles as well. Different reasons may explain such discrepancy such as differences in collagen organization within the stromal layers. The anterior stroma is known to have densely packed and randomly directed interwoven collagen fibers. In contrast, the posterior stroma has a more lamellar organization with collagen fibers arranged parallel to the corneal surface. Moreover, keratocytes, which are transparent except for their nuclei, contribute up to 15% of the stromal volume and are thought to limit backwards scattering. Studies have shown increased keratocyte density in the anterior stroma.

Wang et al. (2004) further elaborated that the gradient of mean corneal densitometry values could be due to a combination of factors, including differences in refractive indices across the cornea, variations in hydration in the axial direction, differences in lamellar structure, and differences in the ratio of keratocytes. Thus, corneal reflectance might result from the interplay of these factors. Our observations align with this perspective, suggesting that the structural differences between the anterior and posterior stroma contribute to the variations in reflectance profiles.

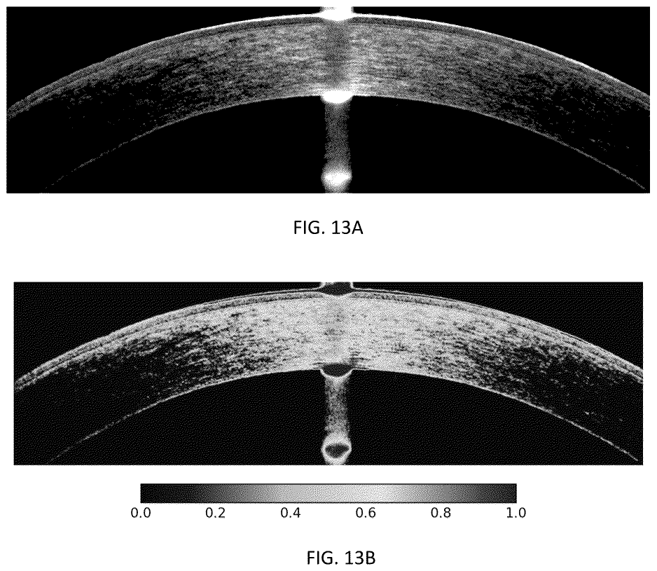

To further illustrate these reflectance differences, an OCT B-scan of the cornea is shown in FIG. 13A and FIG. 13B. FIG. 13A shows a 6-mm wide cornea OCT B-scan in log scale with enhanced contrast for improved visualization. The central flare is a mirror artifact excluded from our image analysis. Parallel organized fibrils are visible in the posterior stroma. FIG. 13 B shows the same OCT B-scan converted to color scale to better illustrate the reflectance. Note the higher reflectance in the posterior stroma, as well as the high reflectance in the epithelium, anterior stroma, and endothelium at higher incidence angles, corroborated by FIG. 12B. As expected, the reflectance profile is non-homogeneous in these images. The stroma exhibits distinct reflectance patterns, with higher intensity observed in the posterior stroma. At higher incidence angles, reflectance is more pronounced in the epithelium, anterior stroma, and endothelium, likely due to a lower directional component. The posterior stroma shows more organized fibrils, visible in FIG. 13A, contributing to a stronger and more directional reflectance. These reflectance differences between the anterior and posterior stroma are consistent with our model.

Previous studies have reported varying results regarding stromal reflectance profiles. Dhubhghaill et al. (2014) and Ning et al. (2024), using Scheimpflug imaging, observed higher densitometry values in the anterior corneal layers compared to the middle and posterior layers. In contrast, Wang et al. (2004), using OCT imaging (wavelength=850 nm), reported that normal corneas exhibited the highest backscatter in the epithelium and posterior stroma, with the anterior stroma showing lower backscatter.

Knighton et al. used an ex vivo approach to study retinal nerve fiber layer reflectance, adjusting the camera and light source to observe directional changes. In contrast, we employed an in vivo method using OCT, leveraging the natural curvature of the cornea to achieve variation in incidence angles. By assuming homogeneity in corneal properties across similar depths, our approach avoids the need for physical manipulation and allows for a more practical, clinically relevant analysis of reflectance patterns.

Our study extends the work of previous researchers by providing quantitative models that account for the angle of incidence in corneal reflectance, a factor not explored in earlier studies. While Scheimpflug imaging has been used to assess corneal densitometry, it suffers from poor axial resolution and inability to segment individual corneal layers. OCT provides higher resolution and layer-specific imaging capabilities, which we utilized to gain detailed insights into the optical behavior of each corneal layer.

Modeling corneal reflectance as a function of incidence angle holds significant clinical potential. Quantitative metrics derived from these models could improve the diagnostic capabilities of OCT by detecting early microstructural changes in the cornea, such as those seen in keratoconus, Fuchs' dystrophy, or corneal scarring. Increased reflectance in specific stromal layers may indicate early stromal haze or scarring, allowing for earlier intervention. Importantly, our findings suggest that non-parametric percentile analysis, such as evaluating the 97^{th} percentile reflectance, should be favored over parametric mean-based models for detecting localized scattering events such as opacities, edema, or haze. This is because parametric mean reflectance models do not account for normal variations in high-intensity scattering events, potentially misclassifying normal high reflectance as opacity, whereas percentile analysis is sensitive to these outliers. Additionally, exploring the relationship between reflectance and directionality patterns and specific corneal diseases could lead to new diagnostic criteria or monitoring tools.

In addition to our modeling, we performed a variance component analysis on the bin-level residuals—defined as the percentage deviation between the measured and model-predicted reflectance—to elucidate the sources of variability in our dataset. In the linear domain, the total normalized variance was 0.42 for the epithelium, 0.56 for the stroma, and 0.49 for the endothelium. Notably, the stroma exhibited the highest overall variability, likely reflecting its complex collagen architecture, whereas the epithelium showed relatively consistent reflectance. Further partitioning revealed that in the epithelium, 31.4% of the variance was attributable to between-patient differences, 6.3% to between-eye differences, and 62.3% to within-eye variability. Similar patterns were observed in the stroma and endothelium—with the endothelium displaying the highest between-patient variability (38.8%) and between-eye variability (10.5%)—suggesting that, in addition to local microstructural heterogeneity, inter-individual differences in the endothelium may be driven by factors such as variations in endothelial cell density or other patient-specific factors, possibly including subclinical endothelial dysfunction or age-related changes

Moreover, both Pearson correlation and two-sample t-test analyses revealed no significant associations between patient age or gender and the normalized residual variance in any layer. It is noteworthy that variance estimates computed in the linear domain are higher than those that would be obtained in the logarithmic domain due to dynamic range compression.

While our study is limited by its retrospective design, its limited sample size, and the exclusive inclusion of normal eyes, it establishes a robust framework for modeling corneal reflectance. Although previous studies have linked corneal densitometry to demographic factors our findings suggest that, when reflectance is quantified on OCT via our approach, such influences are minimal. Importantly, although our cohort was imaged using a single, calibrated OCT system, we believe that—with appropriate calibration and data conversion—the mathematical model presented here can be extended and applied across different OCT devices

In summary, our work enhances the understanding of corneal optical properties by providing a detailed quantitative model of directional reflectance that accounts for depth incidence angle variability. This model holds significant potential for improving OCT-based diagnostics and lays the groundwork for future investigations into the complex interplay between corneal microstructure and optical behavior.

### Example 3

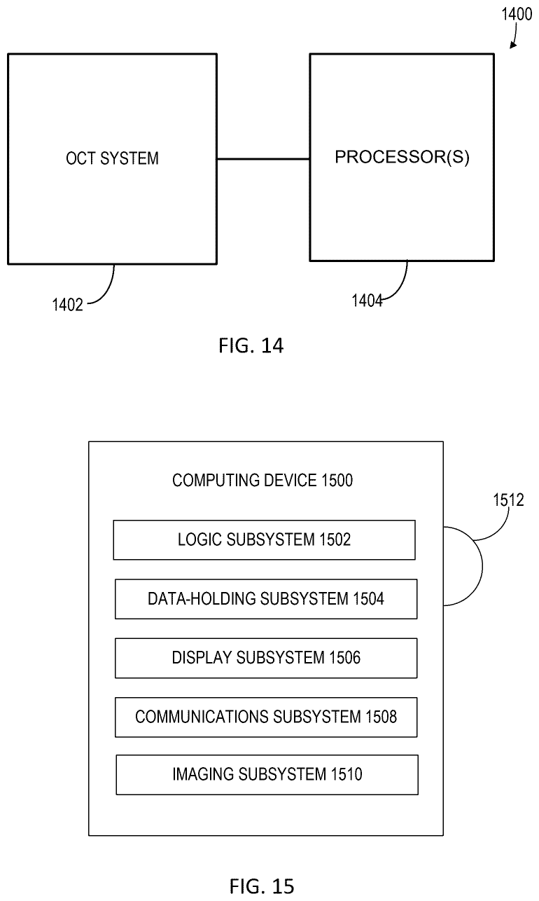

FIG. 14 schematically shows an exemplary system 1400 for OCT image and OCT dataset processing in accordance with various embodiments. System 1400 comprises an OCT system 1402 configured to acquire an OCT image comprising OCT interferograms and one or more processors or computing systems 1404 that are configured to implement the various processing routines described herein. OCT system 1400 may comprise an OCT system suitable for OCT angiography applications, e.g., a swept source OCT system.

In various embodiments, an OCT system may be adapted to allow an operator to perform various tasks. For example, an OCT system may be adapted to allow an operator to configure and/or launch various ones of the herein described methods. In some embodiments, an OCT system may be adapted to generate, or cause to be generated, reports of various information including, for example, reports of the results of scans run on a sample.

In embodiments of OCT systems comprising a display device, data and/or other information may be displayed for an operator. In embodiments, a display device may be adapted to receive an input (e.g., by a touch screen, actuation of an icon, manipulation of an input device such as a joystick or knob, etc.) and the input may, in some cases, be communicated (actively and/or passively) to one or more processors. In various embodiments, data and/or information may be displayed, and an operator may input information in response thereto.

In some embodiments, the methods and processes described herein may be tied to a computing system, including one or more computers. In particular, the methods and processes described herein, e.g., methods 2000 and 2100 described below, may be implemented as a computer application, computer service, computer API, computer library, and/or other computer program product.

FIG. 15 schematically shows a non-limiting computing device 1500 that may perform one or more of the above-described methods and processes. For example, computing device 1500 may represent a processor included in system 1400 described above, and may be operatively coupled to, in communication with, or included in an OCT system or OCT image acquisition apparatus. Computing device 1500 is shown in simplified form. It is to be understood that virtually any computer architecture may be used without departing from the scope of this disclosure. In different embodiments, computing device 1500 may take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1500 includes a logic subsystem 1502 and a data-holding subsystem 1504. Computing device 1500 may optionally include a display subsystem 1506, a communication subsystem 1508, an imaging subsystem 1510, and/or other components not shown in FIG. 15. Computing device 1500 may also optionally include user input devices such as manually actuated buttons, switches, keyboards, mice, game controllers, cameras, microphones, and/or touch screens, for example.

Logic subsystem 1502 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. For example, the one or more processors may comprise physical circuitry programmed to perform various acts described herein. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 1504 may include one or more physical, non-transitory, devices configured to hold data and/or instructions executable by the logic subsystem to implement the herein described methods and processes. When such methods and processes are implemented, the state of data-holding subsystem 1504 may be transformed (e.g., to hold different data).

Data-holding subsystem 1504 may include removable media and/or built-in devices. Data-holding subsystem 1504 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1504 may include devices with one or more of the following characteristics: volatile, nonvolatile, dynamic, static, read/write, read-only, random access, sequential access, location addressable, file addressable, and content addressable. In some embodiments, logic subsystem 1502 and data-holding subsystem 1504 may be integrated into one or more common devices, such as an application specific integrated circuit or a system on a chip.

FIG. 15 also shows an aspect of the data-holding subsystem in the form of removable computer-readable storage media 1512, which may be used to store and/or transfer data and/or instructions executable to implement the herein described methods and processes. Removable computer-readable storage media 1512 may take the form of CDs, DVDs, HD-DVDs, Blu-Ray Discs, EEPROMs, flash memory cards, USB storage devices, and/or floppy disks, among others.

When included, display subsystem 1506 may be used to present a visual representation of data held by data-holding subsystem 1504. As the herein described methods and processes change the data held by the data-holding subsystem, and thus transform the state of the data-holding subsystem, the state of display subsystem 1506 may likewise be transformed to visually represent changes in the underlying data. Display subsystem 1506 may include one or more display devices utilizing virtually any type of technology. Such display devices may be combined with logic subsystem 1502 and/or data-holding subsystem 1504 in a shared enclosure, or such display devices may be peripheral display devices.

When included, communication subsystem 1508 may be configured to communicatively couple computing device 1500 with one or more other computing devices. Communication subsystem 1508 may include wired and/or wireless communication devices compatible with one or more different communication protocols. As non-limiting examples, the communication subsystem may be configured for communication via a wireless telephone network, a wireless local area network, a wired local area network, a wireless wide area network, a wired wide area network, etc. In some embodiments, the communication subsystem may allow computing device 1500 to send and/or receive messages to and/or from other devices via a network such as the Internet.

When included, imaging subsystem 1510 may be used acquire and/or process any suitable image data from various sensors or imaging devices in communication with computing device 1500. For example, imaging subsystem 1510 may be configured to acquire OCT image data, e.g., interferograms, as part of an OCT system, e.g., OCT system 1402 described above. Imaging subsystem 1510 may be combined with logic subsystem 1502 and/or data-holding subsystem 1504 in a shared enclosure, or such imaging subsystems may comprise periphery imaging devices. Data received from the imaging subsystem may be held by data-holding subsystem 1504 and/or removable computer-readable storage media 1512, for example.

### Example 4

The following Example 4 provides a demonstration of an automated corneal opacity detection algorithm with optical coherence tomography (OCT) images based on an incidence-angle- and depth-dependent model of corneal reflectance.

Introduction: Corneal opacity, characterized by the loss of corneal transparency, arises from various etiologies such as trauma, infection, edema, and dystrophies. The cornea owes its transparency to tightly arranged collagen fibrils in the stroma that minimize scatter. Disruption of this arrangement increases scatter and leads to opacity. According to a recent analysis of the American Academy of Ophthalmology's IRIS® Registry, corneal opacities were diagnosed in approximately 6.5% of patients seen in participating ophthalmology practices across the United States between 2013 and 2020, with dystrophies being the most frequently documented etiology. Despite this frequency and impact on vision, current clinical evaluations of corneal opacity often rely on slit-lamp examination, which is inherently subjective and prone to inter-observer variability.

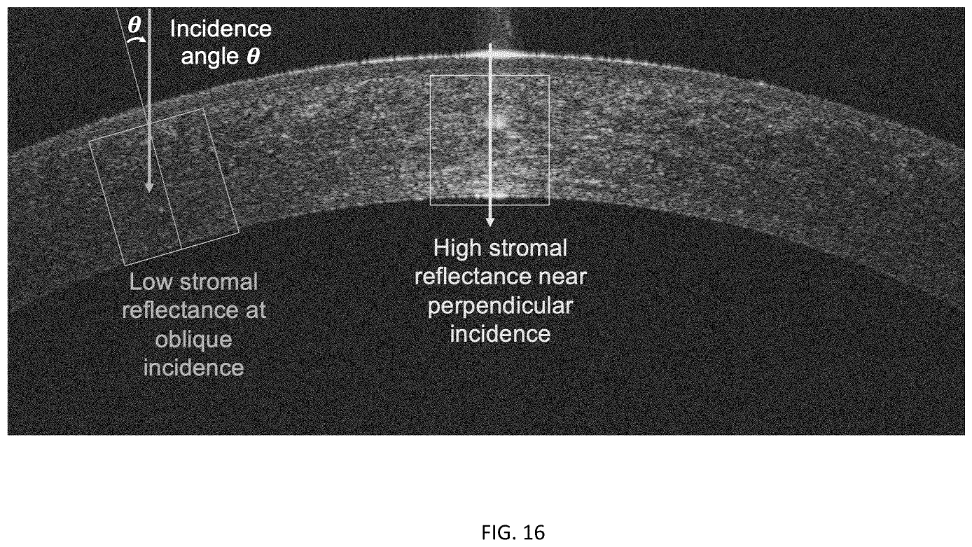

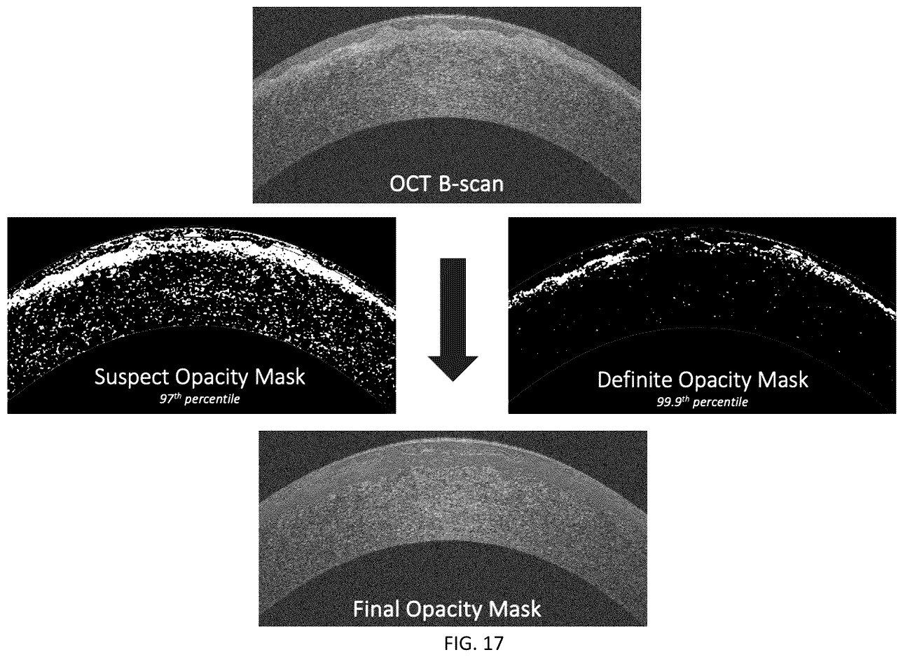

Optical coherence tomography (OCT) offers high-resolution, cross-sectional images of the cornea, enabling quantitative insight into reflectance properties and potential pathological alterations. We have previously characterized the directional reflectance properties of the normal cornea on OCT images (see Example 2 above). We developed mathematical models that quantify reflectance as a function of the angle of beam incidence and tissue depth. These studies revealed that corneal reflectance varies substantially across layers and angles of incidence. FIG. 16 shows an example of the difference in corneal reflectance in the stroma for a perpendicular angle of incidence (high reflectance) versus and oblique angle of incidence (low reflectance). The angle dependence was best fit with a multi-exponential model.

Recent OCT-based methods for quantifying corneal abnormalities have focused on aspects like edema quantification and haze delineation, but they often assume uniform corneal reflectance. Such assumptions neglect natural, layer-specific directional differences and can obscure subtle pathology. To address this gap, we present here a novel algorithm that incorporates an incidence-angle and depth-dependent reflectance baseline to detect and segment corneal opacities more precisely. We then evaluate its performance against physician annotations, highlighting how an incidence angle-aware, depth-specific approach improves objectivity and reliability in corneal opacity detection.

Methods: This retrospective study was conducted at Oregon Health & Science University (OHSU) in Casey Eye Institute and was approved by the OHSU Institutional Review Board. The study adhered to the tenets of the Declaration of Helsinki and was in accord with the Health Insurance Portability and Accountability Act of 1996.

Patient Selection: We analyzed 6-mm-wide anterior segment OCT scans from the Optovue Avanti platform (Visionix USA, Lombard, IL). Patients were classified either as (1) healthy volunteers and LASIK candidates with no abnormal corneal findings confirmed by comprehensive slit-lamp examinations, or (2) those with clinically identified corneal opacities observed on slit-lamp examination.

OCT Calibration: Calibration followed the procedure described in our previous publication, using standard diffuse reflectance targets (12%, 25%, 50%) to map the OCT signal magnitude to absolute reflectance. A linear regression model was used to estimate the 100% reflectance level, and OCT intensities were then normalized to this value to ensure consistent reflectance measurements across imaging sessions and different OCT devices.

Data Preparation: As in our previous work, eight radial B-scan images (640×1024 pixels, scan depth of 2 mm) were extracted from each OCT scan, maintaining their raw, unprocessed, and non-dewarped format so that each axial scan (vertical column of signal values in the OCT B-scan image array) corresponds to the path of the OCT beam through the cornea. A signal roll-off compensation and a low-pass mean filter (rectangular, 3×5 pixels) were applied to each image to reduce speckle noise. Axial scans with central flare artifacts were excluded from subsequent analyses.

Three-Dimensional Incidence Angle Calculation: Incident angles were computed using the three-dimensional (3D) approach described in our previous study. This approach derives the surface normal at each point on the anterior surface from the anterior elevation map and assigns it to the corresponding axial scan. It accounts for the incidence angle of the OCT beam relative to the corneal anterior surface normal both in the plane parallel to the B-scan and in the plane perpendicular to the B-scan.

Binned Reflectance Analysis: To capture layer-specific optical behavior, we performed binned analyses as previously outlined, dividing the epithelium, stroma, and endothelium into uniform sections based on corneal depth and incidence angle. For the stroma, we used 4% depth increments and 0.005-radian angular bins, while the epithelium and endothelium were divided using 10% and 33.3% depth increments, respectively. Pixels from OCT images from multiple patients were then aggregated into bins with matching depth and angle parameters.

Because the distribution of reflectance within the bins is not normal (Gaussian), it is not well characterized by mean and standard deviation parameters. Therefore we adopted a percentile-based approach. Within each bin, we calculated the 97^{th} and 99.9th percentile reflectance values to define thresholds for “suspect” and “definite” opacities, respectively. These upper-tail cut-offs serve as empirical analogues to r-quantiles commonly used in quantile regression.

Curve Fitting: Reflectance values at the 97^{th} and 99.9th percentiles were fitted using exponential functions to model reflectance trends as a function of incidence angle and tissue depth. We applied the same functional form and optimization constraints as previously reported:

$${I}_{k} ( θ ) = {A}_{1 k} {e}^{( - {B}_{1} θ )} + {A}_{2 k} {e}^{( - {B}_{2} θ )} + {A}_{3 k} {e}^{( - {B}_{3} θ )} + C$$

### Where:

Iₖ(θ) represents reflectance in layer k at incidence angle θ.
A₁ₖ, A₂ₖ, and A₃ₖ are the amplitude coefficients for the exponential terms.
B₁ₖ, B₂ₖ, and B₃ₖ are the decay constants.
C is the baseline reflectance term.

The depth increments used for binned reflectance analysis (4% in stroma, 10% epithelium, and 33.3% endothelium), therefore, resulted in 25 curve fits to model reflectance through the depth of the of the stroma, 10 curve fits for the epithelium, and 10 curve fits for the endothelium.

Thresholding and Morphological Fusion: Pixels were labeled as “suspect opacity” if their reflectance exceeded the 97th-percentile threshold T_{S}(x, y), and as “definite opacity” if they exceeded the 99.9th-percentile threshold T_{D}(x, y), based on their specific depth-angle bin. Here, (x, y) denotes the pixel coordinates in the OCT B-scan. We generated the initial binary masks using:

$${M}_{S} = \{ ( x , y ) \vert I ( x , y ) > {T}_{s} ( x , y ) \}$$

$${M}_{D} = \{ ( x , y ) \vert I ( x , y ) > {T}_{D} ( x , y ) \}$$

A mask merging technique then combined these suspect and definite opacity pixels into a single mask using morphological operators. Clusters identified as “definite opacity” were dilated multiple times and merged with any overlapping “suspect opacity” clusters. Symbolically,

$${M}_{final} = {D}^{4} ( E ( {M}_{D} ) ) ⋂ D ( E ( {M}_{S} ) )$$

Where D(⋅) and E(⋅) represent the dilation and erosion operators, respectively. FIG. ° 17 provides a step-by-step illustration of the thresholding and mask fusion workflow. All data analysis, image processing, and computations were performed using MATLAB R2023b (MathWorks Inc., Massachusetts, USA)

Evaluation and Statistical Analysis: The algorithm, trained on scans from healthy volunteers, was evaluated using two separate OCT datasets: (1) normal eyes (control subjects who were LASIK patients scanned prior to surgery) and (2) eyes with clinically identified corneal opacities.

For validation, five trained medical doctors (J. F. A., H. Y., D. S., C. L., I. G.) independently reviewed each OCT scan at both the eye level (opacity present/absent) and at the pixel level (manual segmentation of opacity clusters). Prior to the evaluation, all annotators participated in a brief training session on OCT opacity annotation to standardize their assessment approach. The annotators included two ophthalmology postdoctoral researchers, one ophthalmology resident, one general ophthalmologist, and one cornea specialist, all with prior experience using Avanti OCT scans.

Slit-lamp examination served as the ground truth for eye level classification. For pixel-level validation of the algorithm, “consensus opacity” was defined as pixels labeled by at least 3 out of 5 annotators. For each individual annotator, we used a leave-one-out consensus, defined as pixels marked by ≥3 of the remaining 4 annotators. Agreement between the algorithm (and each individual annotator) and the consensus was quantified using the Dice Similarity Coefficient (DSC):

$$D S C = 2 * \frac{\vert X ⋂ Y \vert}{\vert X \vert + \vert Y \vert}$$

Where X and Y represent the sets of pixels marked as opacities by the algorithm/annotators and the consensus, respectively.

Additionally, we compared the total area of opacity identified by the algorithm and by the annotators to evaluate whether the algorithm under- or over-segments opacities. By incorporating manual annotations, we assessed the algorithm's ability to match or surpass expert performance and also examined the inherent subjectivity of manual OCT annotation.

Statistical comparisons between the algorithm and human annotators were performed using one-sample t-tests to determine if the algorithm's performance metrics were significantly higher or lower than the mean human performance. Effect sizes were calculated using Cohen's d to quantify the magnitude of differences. A p-value <0.05 was considered statistically significant. All statistical analyses were performed using Python (version 3.9) with scipy.stats library.

Results: Demographics: We collected OCT scans from three distinct patient groups: (1) normal eyes for algorithm training, (2) pre-LASIK normal controls, and (3) eyes with various corneal opacities. The training dataset included 95 eyes from 49 volunteers (mean age: 38 years, range: 18-76) with normal corneas. For validation, the pre-LASIK dataset comprised 35 eyes from 35 patients (mean age: 39 years, range: 21-84), and the opacity dataset included 50 eyes from 42 patients (mean age: 56 years, range: 21-85.

The opacity dataset included a range of corneal conditions:
Corneal dystrophies (44% of cases), including granular dystrophy (20%, 10 eyes), Reis-Bücklers dystrophy (8%, 4 eyes), stromal dystrophy (6%, 3 eyes), Fuchs' endothelial dystrophy (4%, 2 eyes), and other hereditary corneal dystrophy (6%, 3 eyes)
Corneal scars (38%, 19 eyes)
Salzmann nodular degeneration (10%, 5 eyes)
Corneal gammopathy (8%, 4 eyes)

Curve Fitting: The optimal mathematical models for each corneal layer were determined based on the approach outlined in our previous study. Reflectance in the epithelium was modeled using a combination of two exponential functions with a constant C. For the stroma and endothelium, three exponential functions with a constant C were required to adequately fit the data.

Opacity Detection and Validation: At the eye level, using slit-lamp examination as the reference, the algorithm achieved an accuracy of 0.93 and an F1-score of 0.94, exceeding the best individual annotator on both accuracy (best 0.88) and F1-score (best 0.89) and outperforming the annotator cohort on average. Its sensitivity and specificity were 0.96 and 0.89, respectively. Human annotators had a mean accuracy of 0.83±0.06 (range: 0.72-0.88), F1-score of 0.85±0.04 (range: 0.80-0.89), sensitivity of 0.84±0.09 (range: 0.74-0.98), and a specificity of 0.80±0.27 (range: 0.34-0.97). Statistical significance testing revealed that the algorithm's performance was significantly superior to human annotators for accuracy (P=0.011, Cohen's d=1.63), F1-score (P=0.003, Cohen's d=2.47), and sensitivity (P=0.019, Cohen's d=1.38), representing large effect sizes. The algorithm's specificity improvement over human annotators (0.89 vs 0.80±0.27) was not statistically significant (P=0.257, Cohen's d=0.32), likely due to the high variability in human specificity measurements.

At the pixel level, the algorithm achieved a DSC of 0.58 when compared to the consensus annotation, whereas individual annotators achieved a mean DSC of 0.71±0.05 (range: 0.667-0.79) when evaluated against their consensus. Statistical analysis revealed that the algorithm's pixel-level performance was significantly lower than human annotators (P=0.002, Cohen's d=2.72), representing a very large effect size.

In terms of total opacity pixel count, the algorithm identified 98% of the pixel count relative to the consensus annotation, indicating that its opacity thresholds were well-calibrated with no significant over- or under-detection of opacity. In contrast, human annotators identified 122%±23% (range: 87%-145%) of the consensus pixel count, reflecting a greater tendency toward over-detection and higher variability among manual evaluations. Statistical analysis revealed that the algorithm's pixel count ratio was significantly lower than human annotators (P=0.037, Cohen's d=−1.07), representing a large effect size.

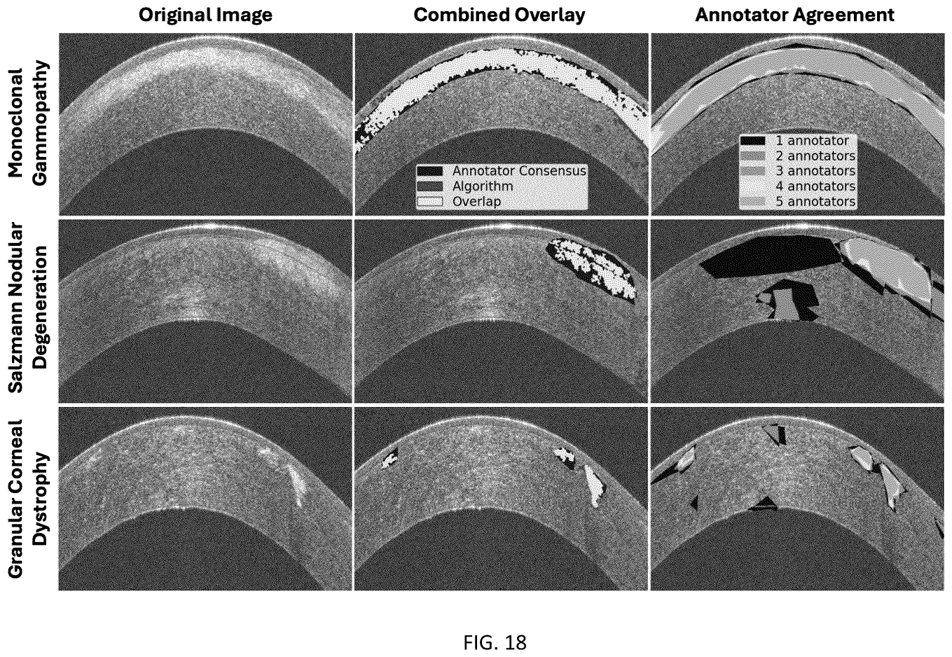

FIG. 18 shows representative corneal OCT B-scans illustrating automated opacity detection in cases of monoclonal gammopathy, Salzmann nodular degeneration, and granular corneal dystrophy. In each row, the algorithm-generated mask (red) overlaps with the consensus annotation (blue; defined as agreement by ≥3 of 5 annotators), with areas of overlap appearing in yellow. The algorithm consistently captured the majority of the consensus region across all three pathologies, while often segmenting with finer granularity. False-positive detections were rare and primarily appeared as small clusters in the peripheral posterior stroma. The inter-annotator agreement heatmaps (right-most panels) revealed that most regions of algorithm-consensus disagreement coincided with areas of annotator disagreement, suggesting that many apparent errors reflect the inherent ambiguity of human interpretation.

Discussion: We developed an automated algorithm for detecting corneal opacities on OCT by applying incidence-angle- and depth-specific reflectance thresholds derived from a normative corneal model. By grounding the algorithm in statistical reflectance distributions, we eliminated subjective bias and established a reproducible framework for quantitative opacity assessment. At the eye level, the algorithm outperformed human annotators, achieving an accuracy of 93%, sensitivity of 96%, and specificity of 89%, underscoring its potential as an objective clinical tool.

A major challenge in developing OCT-based opacity detection algorithms is establishing a reliable ground truth. While a comprehensive slit-lamp examination can confirm the presence or absence of corneal opacities, precise pixel-level segmentation on OCT images remains difficult, particularly for subtle lesions. This ambiguity complicates the creation of annotated datasets suitable for deep learning, which motivated our use of a consensus annotation from five trained ophthalmologists as the most reliable pixel-level reference available.

Existing OCT-based opacity detection algorithms often overlook the fact that corneal reflectance varies, depending on tissue depth and beam incidence angle (FIG. 16). In contrast, our approach explicitly accounts for dependencies by applying depth- and incidence-angle-specific reflectance thresholds, defined at the 97^{th} and 99.9th percentiles of the normal cornea reflectance distribution.

When benchmarked against human annotators using slit-lamp examination as the clinical reference, our algorithm demonstrated superior performance at the eye level. At the pixel level, however, its overlap with the human consensus was low (DSC=0.58) compared to the agreement between individual annotators and their consensus (mean DSC=0.71). Two main factors likely contribute to this discrepancy. First, the algorithm delineates opacity borders with greater granularity than humans (FIG. 18), which can reduce measured overlap with broadly defined manual annotations. Second, human annotators are more prone to false positives in areas of near-perpendicular incidence angle in the anterior and posterior stroma, regions that exhibit inherently higher baseline reflectance in normal corneas. In contrast, the algorithm incorporates depth-dependent reflectance thresholds, helping to suppress these false positives. Supporting this interpretation, areas of disagreement between the algorithm and the consensus often coincide with regions of low inter-annotator agreement (FIG. 18). Additionally, the algorithm occasionally produced small false-positive clusters in the peripheral posterior stroma (FIG. 18), likely due to reduced signal quality and narrower reflectance margins between normal tissue and true opacity in these regions.

Several limitations of our algorithm should be addressed in future studies. Inter-subject variations in corneal reflectivity, potentially influenced by factors such as age, ethnicity, ocular surface conditions, nasal-temporal differences, or imaging quality, may reduce the effectiveness of applying universal thresholds across all eyes. Most false-positive opacity detection by our algorithm occurs in the peripheral cornea, likely due to common occurrence of peripheral degenerative changes coupled with the normally decreased OCT signal intensity in the periphery where the incidence angle is more off-perpendicular. A larger normative dataset that includes more older patients using wide-field 10-mm OCT scans may help minimize this issue. In the interim, limiting the analysis to the central 5-mm corneal zone could temporarily enhance specificity. Additionally, dense central opacities can create shadow artifacts that impair visibility of deeper layers (FIG. 18). Incorporating shadow-compensation algorithms, similar to those established in retinal OCT imaging, may improve accuracy in these challenging cases.

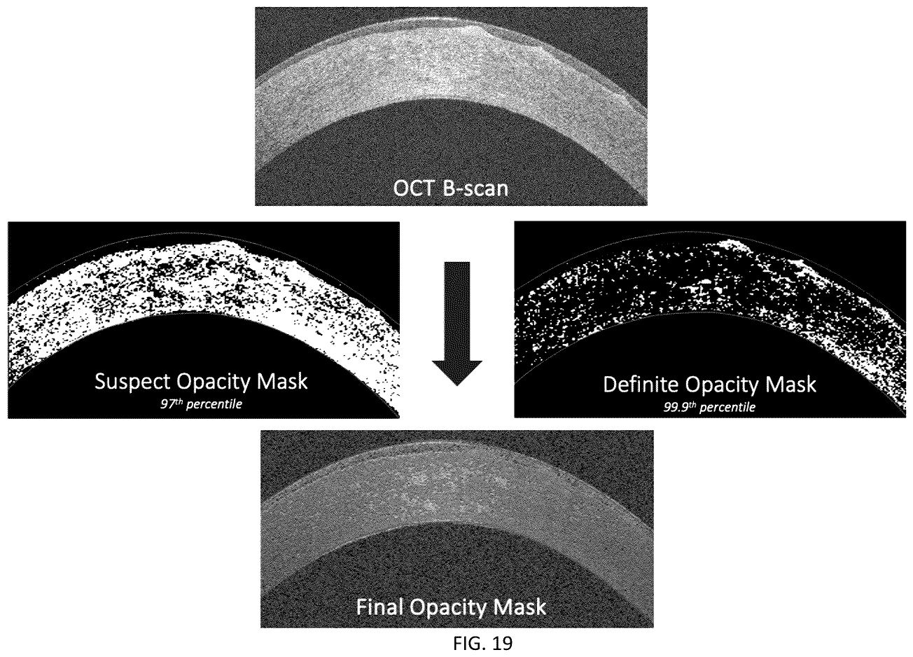

An important clinical application of our algorithm lies in phototherapeutic keratectomy (PTK) planning, where opacity maps could guide the programming of laser ablation. Developing a visual acuity prediction model based on OCT-derived opacity maps would be the logical next step for future research. Such a model would help optimize the ablation pattern to maximize visual improvement without excessive removal of corneal stroma. However, certain dystrophies, such as macular corneal dystrophy, involve both diffuse stromal haze and focal opacities (FIG. 19). In these instances, customized opacity detection thresholds may be necessary to isolate the densest foci of opacities for ablation planning.

Overall, our approach, leveraging incidence-angle- and depth-specific reflectance thresholds, represents a significant advancement toward standardized, objective, and reproducible detection of corneal opacities on OCT, with important implications for both clinical management and future research.

### Example 5

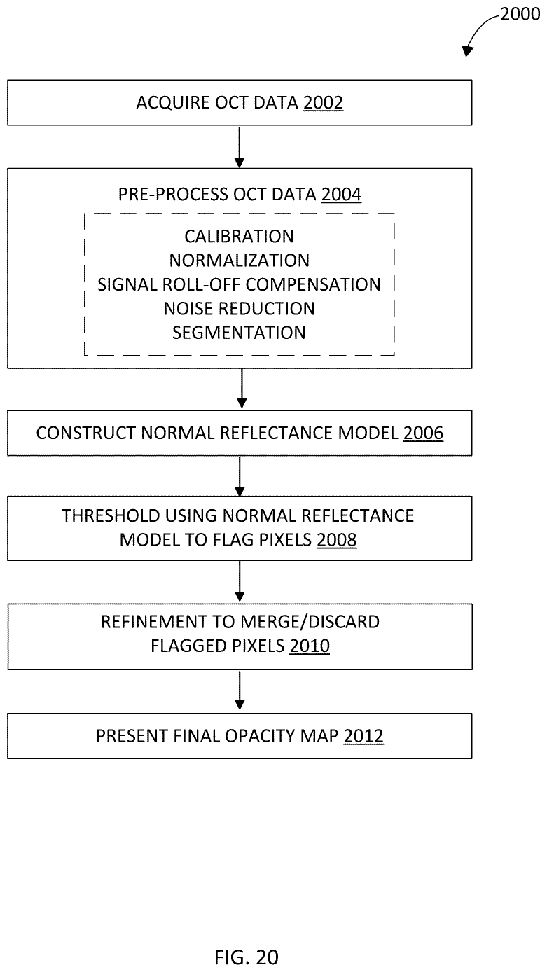

FIG. 20 shows an exemplary method 2000 in flowchart form that may be used to practice certain aspects of the methods described herein. Data acquisition step 2002 may include acquisition of OCT scans from a corneal imaging system or retrieval of previously acquired scan data from a storage device. In various embodiments, the methods may be implemented to receive as input OCT data acquired from different scan patterns such as radial, raster, or spiral scans to capture depthwise A-scan profiles of the cornea.

At 2004, preprocessing of the input OCT data is performed and may include any or all of the following: calibration using standardized diffuse reflective targets, normalization of reflectance values, signal roll-off compensation to address depth-dependent signal attenuation in OCT, noise reduction (e.g., low pass filtering), and segmentation of the cornea image data into anatomic layers.

At step 2006, construction of the normal reflectance model is performed. For each layer and sublayer depth, reflectance is measured for a range of incidence angles. Incidence angles may be determined as the angle between the OCT scan beam direction (e.g., the A-scan direction) and the normal to the corneal surface, and may further include accounting of the refraction of the OCT beam at the air-cornea interface. Empirical or curve fitting methods are then used to define a reflectance function that characterize how reflectance changes with incidence angle and depth. The coefficients that define the reflectance function are stored for reference.

At step 2008 a thresholding procedure is performed to detect opacity within the corneal OCT data. As part of this procedure, each pixel is mapped to its layer, depth, and incidence angle. The normal (i.e. non-pathologic) reflectance expected at that location is obtained from the model and associated stored coefficients. If the observed reflectance exceeds a chosen high percentile (e.g., 97^{th} or 99.9th) relative to the model distribution, that location is flagged as an opacity.

After thresholding to identify and flag pixels as opacities, a refinement step at 2010 may be performed to merge or discard sets of flagged pixels. For example, morphological operations such as dilation and erosion can be used to merge contiguous areas exceeding a prescribed threshold. At this step, small, isolated regions under a minimum prescribed size may be discarded (i.e., not categorized as opacities), while large clusters of high-intensity pixels may be combined into and presented as a final opacity map 2012.

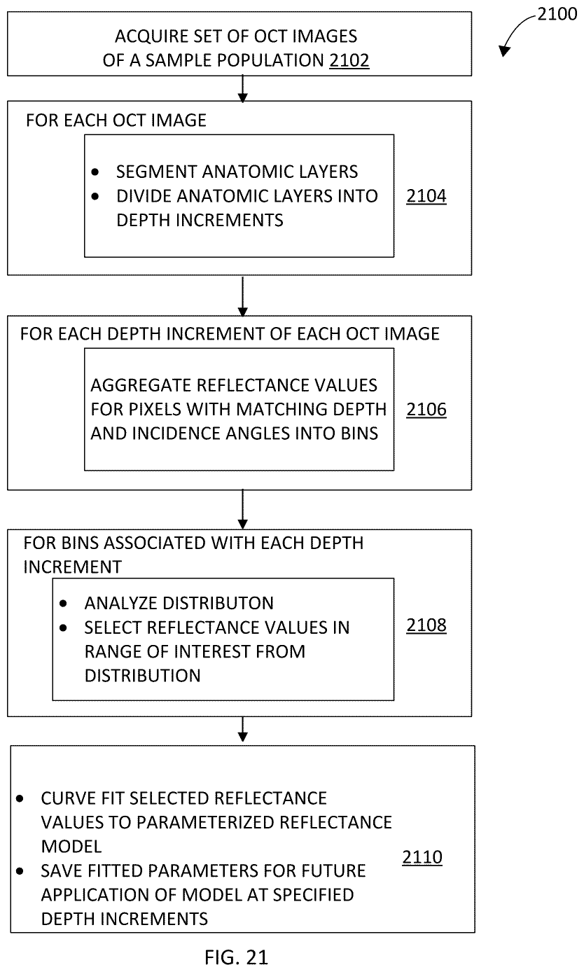

FIG. 21 shows another flowchart of a non-limiting example method 2100 for generating a reflectance model from population data in accordance with the methods described herein. Such a reflectance model may be used, in some embodiments, to establish layer- and depth-specific threshold values to detect abnormally high reflectance values which may indicate localized corneal opacities. In the particular example shown here, the approach is useful cases where the population reflectance exhibits a non-gaussian distribution.

At 2102, a set of OCT images are acquired from a representative population sample. At step 2104, for each OCT images in the population, the cornea is segmented into anatomic layers and then each anatomic layer is further divided depth-wise into a series of depth increments. As described throughout this disclosure, each pixel within the segmented anatomic layers (and, therefore, within each depth increment) is associated with a depth value and an angle of incidence value. Next, at 2106, measured reflectance values from each depth increment having matching depth and incidence angle are aggregated into bins. This aggregation is carried out for corresponding depth increments across the sample population. At 2108, the distributions in each bin are analyzed and a subset of binned reflectance values are selected from a distribution range of interest for further analysis. For example, reflectance values from the upper-tail of the distribution (for example, at the 99^{th} percentile level) may be selected to develop a threshold reflectance model to detect abnormally high reflectance values in a OCT image. At 2110, the selected reflectance values are fit to a parameterized mathematical model to recover a set of fitting parameter values (e.g. model coefficients, constants, weights, etc.). These fitting parameter values can be saved for subsequent use in a reflectance model for thresholding or other analysis within a given depth increment.

This above description and following examples and the accompanying figures explain possible embodiments of the disclosed systems & methods. They are not intended to limit the implementation or practice of the disclosed systems and methods to any particular form but rather to illustrate the principles that may be applied in numerous ways without departing from the scope of the claims.

1. A method comprising:
obtaining an OCT dataset of a cornea, the OCT dataset comprising a plurality of A-scans, each of the A-scans comprising a plurality of pixels extending depth-wise through the cornea and each having a measured signal value;
segmenting the pixels of the OCT dataset into two or more anatomic layers;
dividing each of the two or more anatomic layers into a plurality of depth increments;
associating, for each of the pixels within the two or more anatomic layers, an angle of incidence and a depth with the measured signal value;
for each of the depth increments, identifying a first set of pixels whose signal values exceed a threshold calculated using a reflectance model that is dependent on anatomic layer, angle of incidence, and depth; and
generating a map of the first set of pixels.

2. The method of claim 1, further comprising:
identifying a second set of pixels within each of the depth increments whose signal values exceed a higher threshold calculated using the reflectance model; and
identifying, on the map of the first set of pixels, pixels that are connected to the second set of pixels, thereby generating an opacity map.

3. The method of claim 1, wherein the two or more anatomic layers are selected from the group consisting of epithelium, stroma, endothelium, one or more subdivided layers within the epithelium, one or more subdivided layers within the stroma, and one or more subdivided layers within the endothelium.

4. The method of claim 1, wherein the angle of incidence for each of the plurality of A-scans is calculated as the angle between a normal to the anterior corneal surface at said A-scan and the direction of an OCT beam incident upon the anterior corneal surface.

5. The method of claim 1, wherein the depth is expressed as a percentage of the thickness of the selected anatomic layer.

6. The method of claim 1, further comprising:
calibrating the signal values of the OCT dataset of the cornea relative to samples of known reflectance values;
adjusting the signal values to compensate for depth-dependent signal roll-off; and
applying a spatial filter to reduce speckle noise.

7. The method of claim 1, wherein the reflectance model is based on a collection of OCT corneal reflectance data from a sample of normal human population.

8. The method of claim 7, wherein the reflectance model is determined from reflectance values corresponding to a selected percentile or upper-tail portion of the normative population reflectance distribution.

9. The method of claim 1, wherein the reflectance model is characterized by a set of fitted model parameters that are specific to each depth increment.

10. A system comprising:
a OCT system;
a logic subsystem; and
a data holding subsystem comprising non-transitory machine-readable instructions stored thereon that are executable by the logic subsystem to:
obtain an OCT dataset of a cornea, the OCT dataset comprising a plurality of A-scans, each of the A-scans comprising a plurality of pixels extending depth-wise through the cornea and each having a measured signal value;
segment the pixels of the OCT dataset into two or more anatomic layers;
divide each of the two or more anatomic layers into a plurality of depth increments;
associate, for each of the pixels within the two or more anatomic layers, an angle of incidence and a depth;
for each of the depth increments, identify a first set of pixels whose signal values exceed a threshold calculated using a reflectance model that is dependent on anatomic layer, angle of incidence, and depth; and
generate a map of the first set of pixels.

11. The system of claim 10, further comprising instructions to:
identify a second set of pixels within each of the depth increments whose signal values exceed a higher threshold calculated using the reflectance model; and
identify, on the map of the first set of pixels, pixels that are connected to the second set of pixels, to generate an opacity map.

12. The system of claim 10, wherein the two or more anatomic layers are selected from the group consisting of epithelium, stroma, endothelium, one or more subdivided layers within the epithelium, one or more subdivided layers within the stroma, and one or more subdivided layers within the endothelium.

13. The system of claim 10, wherein the angle of incidence for each of the plurality of A-scans is calculated as the angle between a normal to the anterior corneal surface at said A-scan and the direction of an OCT beam incident upon the anterior corneal surface

14. The system of claim 10, wherein the depth is expressed as a percentage of the thickness of the selected anatomic layer.

15. The system of claim 10, further comprising:
calibrating the signal values of the OCT dataset of the cornea relative to samples of known reflectance values;
adjusting the signal values to compensate for depth-dependent signal roll-off; and
applying a spatial filter to reduce speckle noise.

16. The system of claim 10, wherein the reflectance model is based on a collection of OCT corneal reflectance data from a sample of normal human population.

17. The system of claim 10, wherein the reflectance model is determined from reflectance values corresponding to a selected percentile or upper-tail portion of the normative population reflectance distribution.

18. The system of claim 17, wherein the reflectance model is characterized by a set of fitted model parameters that are specific to each depth increment.
